(19) United States
(12) Patent Application Publication
(10) Pub. No.: US 2025/0195589 A1
(43) Pub. Date: Jun. 19, 2025

(54) VAGINAL LIVE BIO-THERAPEUTIC COMPOSITIONS AND METHODS OF USE THEREOF

(71) Applicants: The General Hospital Corporation, Boston, MA (US); University of Maryland, Baltimore, Baltimore, MD (US); Centre for the AIDS Programme of Research in South Africa, Durban, KwaZulu-Natal (ZA); University of Cape Town, Rondebosch (ZA)

(72) Inventors: Douglas Kwon, Somerville, MA (US); Jacques Ravel, Laurel, MD (US); Caroline Morrissey Mitchell, Lincoln, MA (US); Michael France, Catonsville, MD (US); Jo-Ann Passmore, Rondebosch (ZA); Sinaye Ngcapu, KwaZulu-Natal (ZA); Fatima Hussain, Somerville, MA (US); Anna-Ursula Happel, Cape Town (ZA)

(21) Appl. No.: 18/842,931

(22) PCT Filed: Mar. 1, 2023
(86) PCT No.: PCT/US2023/063490

§ 371 Date: Aug. 30, 2024

Related U.S. Application Data
(60) Provisional application No. 63/382,704, filed on Nov. 7, 2022.
(60) Provisional application No. 63/315,503, filed on Mar. 1, 2022.

(51) Int. Cl. A61K 35/747

(52) U.S. Cl. CPC A61K 35/747

(57) ABSTRACT

Described herein are vaginal live biotherapeutic compositions for treatment of vaginal dysbiosis or conditions/disorders associated with vaginal dysbiosis. Also disclosed herein are methods for treating vaginal dysbiosis in an individual in need thereof, methods for reducing the risk of vaginal dysbiosis in an individual in need thereof, and methods for restoring Lactobacillus dominated microbiota in an individual in need thereof.

### CLAIM OF PRIORITY

This application claims the benefit of U.S. Provisional Patent Applications Ser. Nos. 63/315,503, filed on Mar. 1, 2022, and 63/382,704, filed on Nov. 7, 2022. The entire contents of the foregoing are hereby incorporated by reference.

### SEQUENCE LISTING

This application contains a Sequence Listing that has been submitted electronically as an XML file named 29539-0653WO1_SL_ST26.xml. The XML file, created on Feb. 27, 2023, is 37,123,905 bytes in size. The material in the XML file is hereby incorporated by reference in its entirety.

### BACKGROUND OF THE INVENTION

Vaginal colonization with a Lactobacillus-dominant microbial community is associated with lower risk for preterm birth, HIV acquisition, HPV persistence and cervical dysplasia.¹⁻⁵ Determinants of vaginal microbial community stability and Lactobacillus dominance are unknown—is it host factors, environmental influences or microbial inoculum? Some women have an optimal, stable, Lactobacillus-dominant microbiome, while others fluctuate between this “optimal” state and a more diverse community such as the vaginal microbial communities of women suffering from bacterial vaginosis (BV), putting them at higher risk of adverse outcomes.^{6, 7} The absence of an animal model for the human vaginal microbiota means that untangling the many potential drivers of vaginal microbial colonization is difficult.⁸

Worldwide, 25-30% of women have BV, which is associated with increased risk for preterm birth, miscarriage, HIV acquisition and cervical dysplasia-conditions leading to billions of dollars in health care costs annually.^{1, 9} Furthermore, the prevalence of a diverse, non-Lactobacillus dominant vaginal microbiota is higher in sub-Saharan Africa than the US^{2,3}. Within US cohorts, Black women are more likely to have a diverse vaginal microbiota³. L. crispatus dominance is much less likely in young sub-Saharan African women, as well as US Black women. The reason for these differences is unknown.

Although antibiotic treatment of BV reduces the absolute burden of BV-associated microbes, it has a one-month cure rate of 40-70%, demonstrating rapid and frequent recurrence.¹⁰⁻¹² BV is characterized both by increased diversity and quantity of species in the vaginal ecosystem, and by the absence of vaginal Lactobacillus colonization. The two most commonly identified vaginal Lactobacillus species are L. crispatus (considered beneficial) and L. iners (considered less beneficial and less stable than L. crispatus).^{1, 13, 14} Antibiotics reduce the burden of pathologic, BV-associated anaerobes and short-term cure rates are as high as 70%, but treatment has high rates of relapse.

After antibiotic treatment, the vaginal community is more often dominated by L. iners than L. crispatus.¹⁵⁻¹⁷ Oral probiotic strategies to recolonize with beneficial Lactobacillus species have had small or no benefit.^{15, 18, 19} Delivering exogenous L. crispatus to the vagina using, one, single-strain L. crispatus live biotherapeutic product (Lactin-V) has been studied. In a Phase 2a trial, Lactin-V colonized 50% of treated women after 5 daily doses, but 18 days later colonization dropped to 44%.^{20, 21} In the Phase 2b trial, after 5 daily doses and then twice weekly dosing for 11 weeks, 79% of women were colonized in the final week of treatment, but this dropped to 48% after an additional 12 weeks with no treatment.²² Women treated with Lactin-V had a significantly lower risk for recurrent BV at both 12 and 24 weeks. These results suggest that 1) vaginal inoculation with L. crispatus can decrease recurrent dysbiosis, and 2) inoculating a single strain is insufficient to establish durable colonization of 1 . . . crispatus in some women.

An effective clinical intervention to durably shift the vaginal microbiota to Lactobacillus dominance—and ideally, L. crispatus dominance—would allow improved clinical treatment of women with recurrent BV, which is associated with adverse health outcomes and has a significant negative impact on quality of life.²³ In addition, if such an intervention promoted durable L. crispatus dominance, it could potentially be used to decrease rates of adverse health outcomes, including of preterm birth,^{24, 25} HIV acquisition,¹ cervical dysplasia,^{2, 3} post-operative gynecologic infections²⁶ and increase the success of in vitro fertilization²⁷ and spontaneous conception, all of which would provide a significant public health benefit.

### SUMMARY OF THE INVENTION

It has now been determined that optimal combinations of L. crispatus isolates can be formulated into a live biotherapeutic composition and administered to promote a vaginal microbial community dominated by beneficial Lactobacilli (i.e., to establish non-L. iners Lactobacillus dominance) which is crucial to reducing risk of acquiring recurrent BV.

Disclosed herein are methods for treating or reducing the risk of vaginal dysbiosis in an individual in need thereof, said method including administering a vaginal live bio-therapeutic composition to the individual, wherein the vaginal live bio-therapeutic composition comprises two or more Lactobacillus crispatus strains selected from FF00051, C0022A1, C0175A1, C0059E1, FF00018, UC101_2_33 and combinations thereof, thereby treating vaginal dysbiosis in the individual. In some embodiments, the vaginal live bio-therapeutic composition comprises two Lactobacillus crispatus strains selected from FF00051, C0022A1, C0175A1, C0059E1, FF00018, UC101_2_33. In some embodiments, the vaginal live bio-therapeutic composition comprises three Lactobacillus crispatus strains selected from FF00051, C0022A1, C0175A1, C0059E1, FF00018, UC101_2_33. In some embodiments, the vaginal live bio-therapeutic composition comprises four Lactobacillus crispatus strains selected from FF00051, C0022A1, C0175A1, C0059E1, FF00018, UC101_2_33. In some embodiments, the vaginal live bio-therapeutic composition comprises five Lactobacillus crispatus strains selected from FF00051, C0022A1, C0175A1, C0059E1, FF00018, UC101_2_33. In some embodiments, the vaginal live bio-therapeutic composition comprises the Lactobacillus crispatus of FF00051, C0022A1, C0175A1, C0059E1, FF00018, UC101_2_33. In some embodiments, the two or more L. crispatus strains are selected from 122010_1999_16, C0022A1, C0175A1, C0028A1, C0006A1, FF00018, UC101_2_33, UC119_2_11, 185329_1999_17, C0112A1, FF00064, FF00051, and combinations thereof.

In some embodiments, the vaginal live bio-therapeutic composition also includes at least one additional (e.g., one additional, two additional, three additional, four additional, five additional, six additional, seven additional, eight additional, or nine additional) Lactobacillus crispatus strain(s). In some embodiments, the vaginal live bio-therapeutic composition also includes at least one additional (e.g., one additional, two additional, three additional, four additional, five additional, six additional, seven additional, eight additional, or nine additional) Lactobacillus crispatus strain originally isolated from a vaginal tissue sample obtained from a female individual (or individual with a vagina) having a stable Lactobacillus-dominated vaginal microbiome. In some embodiments, the at least one additional (e.g., one additional, two additional, three additional, four additional, five additional, six additional, seven additional, eight additional, or nine additional) Lactobacillus crispatus strain is selected from C0028A1, C0006A1, C0112A1, FF00004, UC119_2_11, FF00064, 122010_1999_16, 185329_1999_17 and FF00072, and combinations thereof.

In some embodiments, the vagina dysbiosis is bacterial vaginosis (BV).

Also disclosed herein are vaginal live bio-therapeutic compositions that include two or more Lactobacillus crispatus strains selected from FF00051, C0022A1, C0175A1, C0059E1, FF00018, UC101_2_33, and combinations thereof. In some embodiments, the vaginal live bio-therapeutic composition comprises two Lactobacillus crispatus strains selected from FF00051, C0022A1, C0175A1, C0059E1, FF00018, UC101_2_33. In some embodiments, the vaginal live bio-therapeutic composition comprises three Lactobacillus crispatus strains selected from FF00051, C0022A1, C0175A1, C0059E1, FF00018, UC101_2_33. In some embodiments, the vaginal live bio-therapeutic composition comprises four Lactobacillus crispatus strains selected from FF00051, C0022A1, C0175A1, C0059E1, FF00018, UC101_2_33. In some embodiments, the vaginal live bio-therapeutic composition comprises five Lactobacillus crispatus strains selected from FF00051, C0022A1, C0175A1, C0059E1, FF00018, UC101_2_33. In some embodiments, the vaginal live bio-therapeutic composition comprises the Lactobacillus crispatus strains of FF00051, C0022A1, C0175A1, C0059E1, FF00018, UC101_2_33. In some embodiments, the two or more L. crispatus strains are selected from 122010_1999_16, C0022A1, C0175A1, C0028A1, C0006A1, FF00018, UC101_2_33, UC119_2_11, 185329_1999_17, C0112A1, FF00064, FF00051, and combinations thereof.

In some embodiments, the composition also includes at least one additional (e.g., one additional, two additional, three additional, four additional, five additional, six additional, seven additional, eight additional, or nine additional) Lactobacillus crispatus strain originally isolated from a vaginal tissue sample obtained from a female individual (or individual with a vagina) having a stable Lactobacillus-dominated vaginal microbiome. In some embodiments, the at least one additional (e.g., one additional, two additional, three additional, four additional, five additional, six additional, seven additional, eight additional, or nine additional) Lactobacillus crispatus strain is selected from C0028A1, C0006A1, C0112A1, FF00004, UC119_2_11, FF00064, 122010_1999_16, 185329_1999_17, and FF00072, and combinations thereof.

Also provided herein are compositions comprising comprise two or more (e.g., three or more, four or more, five or more, six or more, seven or more, eight or more, nine or more, ten or more, eleven or more, twelve or more, thirteen or more, fourteen or more, or fifteen or more) Lactobacillus crispatus strains or “isolates” including, but not limited to, Lactobacillus crispatus strains 122010_1999_16, C0022A1, C0175A1, C0059E1, C0028A1, C0006A1, FF00018, UC101_2_33, UC119_2_11, 185329_1999_17, C0112A1, FF00004, FF00064, FF00051, FF00072, and combinations thereof, and methods of use thereof.

In some embodiments, the composition also includes at least one of lactic acid, maltose, glycogen, glutamine and magnesium citrate or combinations thereof. In some embodiments, the composition further comprises at least one mucoadhesive. In some embodiments, the composition also includes at least one antimicrobial peptide.

Also disclosed herein are methods for restoring Lactobacillus dominated microbiota in an individual in need thereof, said method including administering a vaginal live bio-therapeutic composition to the individual, wherein the composition comprises two or more Lactobacillus crispatus strains selected from FF00051, C0022A1, C0175A1, C0059E1, FF00018, UC101_2_33, and combinations thereof, thereby restoring Lactobacillus dominated microbiota in the individual. In some embodiments, the vaginal live bio-therapeutic composition also includes at least one additional (e.g., one additional, two additional, three additional, four additional, five additional, six additional, seven additional, eight additional, or nine additional) Lactobacillus crispatus strain originally isolated from a vaginal tissue sample obtained from a female individual (or individual with a vagina) having a stable Lactobacillus-dominated vaginal microbiome. In some embodiments, the at least one additional (e.g., one additional, two additional, three additional, four additional, five additional, six additional, seven additional, eight additional, or nine additional) Lactobacillus crispatus strain is selected from C0028A1, C0006A1, C0112A1, FF00004, UC119_2_11, FF00064, 122010_1999_16, 185329_1999_17 and FF00072, and combinations thereof.

In some embodiments, additional active agents are included in the compositions and methods described herein. For example, a cysteine uptake inhibitor, an antibiotic treatment against BV, or a cysteine uptake inhibitor and an antibiotic treatment against BV can be included.

In some embodiments, additional active agents are included in the compositions described and used herein. For example, a cysteine uptake inhibitor, an antibiotic treatment against BV, or a cysteine uptake inhibitor and an antibiotic treatment against BV. In some embodiments, a cysteine uptake inhibitor can prevent a bacterial cell (e.g., a cell of a bacterial species contributing to BV), from transporting cysteine into the cell from outside the cell. In some embodiments, a cysteine uptake inhibitor can selectively inhibit Lactobacillus iners growth in vitro or in vivo. Non-limiting cysteine uptake inhibitors can include S-methyl-L-cysteine (SMC) and seleno-DL-cysteine (SDLC). (See, for example, Bloom S M et al. Nat. Microbiol., (2022) March; 7 (3): 434-450, which is incorporated in its entirety.)

Other features and advantages of the invention will be apparent from the Detailed Description, and from the claims. Thus, other aspects of the invention are described in the following disclosure and are within the scope of the invention.

### BRIEF DESCRIPTION OF THE DRAWINGS

The following Detailed Description, given by way of example, but not intended to limit the invention to specific embodiments described, may be understood in conjunction with the accompanying figures, incorporated herein by reference.

FIGS. 1A-1B are graphs that depict similar and low levels of IL8 between all Lactobacillus crispatus strains using conditioned media on End1 cells. JV-V01 is a control strain.

FIG. 2 is a graph that depicts cytokine induction by Lactobacillus crispatus strains using conditioned media on End1 cells. PIC=Poly(I:C), a TLR3 agonist and a positive control.

FIGS. 3A-3B are graphs that depict cytokine induction by Lactobacillus crispatus strains co-cultured with End1 cells. PIC=Poly(I:C), a TLR3 agonist and a positive control. KSF=Keratinocyte serum free medium, a negative control. JV-V01 (also known as HMP ID 506) and 33197 (also known as ATCC strain VPI 7635) are control strains.

FIGS. 4A-4H depict antimicrobial properties of various Lactobacillus crispatus strains and combinations thereof. Antimicrobial properties were determined using cross-streaking assays.

### DETAILED DESCRIPTION OF THE INVENTION

### Definitions

Unless otherwise defined, all technical and scientific terms used herein have the same meaning as commonly understood by one of ordinary skill in the art to which this invention belongs. In case of conflict, the present application, including definitions will control.

A “individual” is a vertebrate, including any member of the class Mammalia, including humans, domestic and farm animals, and zoo, sports or pet animals, such as mouse, rabbit, pig, sheep, goat, cattle and higher primates.

As used herein, the terms “treat,” “treating,” “treatment,” and the like refer to reducing or ameliorating a vaginal dysbiosis disorder (e.g., BV), and/or to ameliorate at least one symptom of the disorder associated with vaginal dysbiosis disorders (e.g., cervical dysplasia, preterm birth, acquiring sexually transmitted infections, post-operative gynecological infections, or unsuccessful in vitro fertilization and spontaneous conception) in an individual in need thereof. It will be appreciated that, although not precluded, treating a disorder or condition does not require that the disorder, condition or symptoms associated therewith be completely eliminated.

By “effective amount” or a “therapeutically effective amount” is meant the amount Live Biotherapeutic Products (LBPs) that produce the desired therapeutic response (i.e., producing, restoring and/or enhancing vaginal function) and in specific embodiments, “effective amount” refers to the amount of vaginally administered LBP needed to achieve vaginal colonization with a Lactobacillus-dominant microbial community).

By “vaginal dysbiosis” is meant displacement of optimal vaginal microbiota (e.g., dominated by L. crispatus) by diverse strict and facultative anaerobic microbiota (Gardnerella vaginalis, Atopobium vaginae, Prevotella bivia, Megasphaera spp., among others) and the absence or low abundance of Lactobacillus species.

By “isolated” is meant a material that is free to varying degrees from components which normally accompany it as found in its native state. “Isolate” denotes a degree of separation from original source or surroundings.

Unless specifically stated or clear from context, as used herein, the term “about” is understood as within a range of normal tolerance in the art, for example within 2 standard deviations of the mean. “About” is understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Other definitions appear in context throughout this disclosure.

### Compositions Comprising Live Biotherapeutic Products (LBPs)

Described herein are compositions and methods of treatment or reducing the risk of a vaginal dysbiosis disorder or a disorder associated with a vaginal dysbiosis disorder (e.g., bacterial vaginosis (BV), cervical dysplasia, reduction in the risk of preterm birth, reduction in the risk of acquiring sexually transmitted infections, reducing the risk of infections associated with oral antibiotics, reducing the risk of post-operative gynecological infections, or reducing the risk of unsuccessful in vitro fertilization and/or spontaneous conception) using vaginally administered live biotherapeutic products. Methods for reducing the risk of a disorder can be used, e.g., in subjects who are at risk of developing a vaginal dysbiosis disorder or a disorder associated with a vaginal dysbiosis disorder. Subjects who are at risk include individuals with vaginas who have a history of such disorders, recurrence of such disorders, are undergoing gynecological surgery, who are pregnant, undergoing in vitro fertilization, or trying to conceive, or are taking oral antibiotics.

Vaginal Live Biotherapeutic Products (LBPs) as described herein can comprise two or more (e.g., three or more, four or more, five or more, six or more, seven or more, eight or more, nine or more, ten or more, eleven or more, twelve or more, thirteen or more, fourteen or more, or fifteen or more) Lactobacillus crispatus strains or “isolates” including, but not limited to, Lactobacillus crispatus strains 122010_1999_16, C0022A1, C0175A1, C0059E1, C0028A1, C0006A1, FF00018, UC101_2_33, UC119_2_11, 185329_1999_17, C0112A1, FF00004, FF00064, FF00051, FF00072, and combinations thereof.

In some embodiments, the two or more L. crispatus strains are selected from 122010_1999_16, C0022A1, C0175A1, C0028A1, C0006A1, FF00018, UC101_2_33, UC119_2_11, 185329_1999_17, C0112A1, FF00064, FF00051, and combinations thereof.

In some embodiments, the two or more L. crispatus strains are selected from C0022A1, C0175A1, C0059E1, C0028A1, C0006A1, C0112A1, and combinations thereof.

In some embodiments, the two or more L. crispatus strains are selected from 122010_1999_16, FF00018, UC101_2_33, UC119_2_11, 185329_1999_17, FF00004, FF00064, FF00051, FF00072, and combinations thereof.

In some embodiments, the two or more L. crispatus strains are selected from 122010_1999_16, C0022A1, C0175A1, C0059E1, C0028A1, C0006A1, FF00018, 185329_1999_17, C0112A1, FF00004, FF00064, FF00051, FF00072, and combinations thereof.

In some embodiments, the two or more L. crispatus strains are selected from C0022A1, C0175A1, C0059E1, C0028A1, C0006A1, FF00018, UC101_2_33, UC119_2_11, C0112A1, FF00004, FF00064, FF00051, FF00072, and combinations thereof.

In some embodiments, the two or more L. crispatus strains are selected from 122010_1999_16, C0022A1, C0175A1, C0059E1, C0028A1, C0006A1, UC101_2_33, UC119_2_11, 185329_1999_17, C0112A1, and combinations thereof.

In some embodiments, the strains comprise a six-strain consortium comprising or consisting of L. crispatus strains C0022A1, C0175A1, C0059E1, FF00018, FF00051 and UC101_2_33.

In some embodiments, the strains comprise a six-strain consortium comprising or consisting of L. crispatus strains C0022A1, C0175A1, C0112A1, FF00051, FF00018 and UC101_2_33.

In some embodiments, the strains comprise a five-strain consortium comprising or consisting of L. crispatus strains C0022A1, C0175A1, C0059A1, UC119_2_14 and UC101_2_33.

In some embodiments, the strains comprise a five-strain consortia comprising or consisting of L. crispatus strains C0175A1, C0112A1, FF00051, FF00018 and FF00064.

In some embodiments, the strains comprise a five-strain consortia comprising or consisting of L. crispatus strains C0006A1, C0112A1, C0175A1, FF00018, and FF00064.

In some embodiments, the strains comprise or consist of L. crispatus strains 122010_1999_16, C0022A1, C0175A1, C0028A1, C0006A1, FF00018, UC101_2_33, UC119_2_11, 185329_1999_17, C0112A1, FF00064, and FF00051.

In some embodiments, the strains comprise or consist of L. crispatus strains C0022A1, C0175A1, C0028A1, C0006A1, and C0112A1.

In some embodiments, the strains comprise or consist of L. crispatus strains 122010_1999_16, C0022A1, C0175A1, C0028A1, C0006A1, FF00018, 185329_1999_17, C0112A1, FF00064, and FF00051.

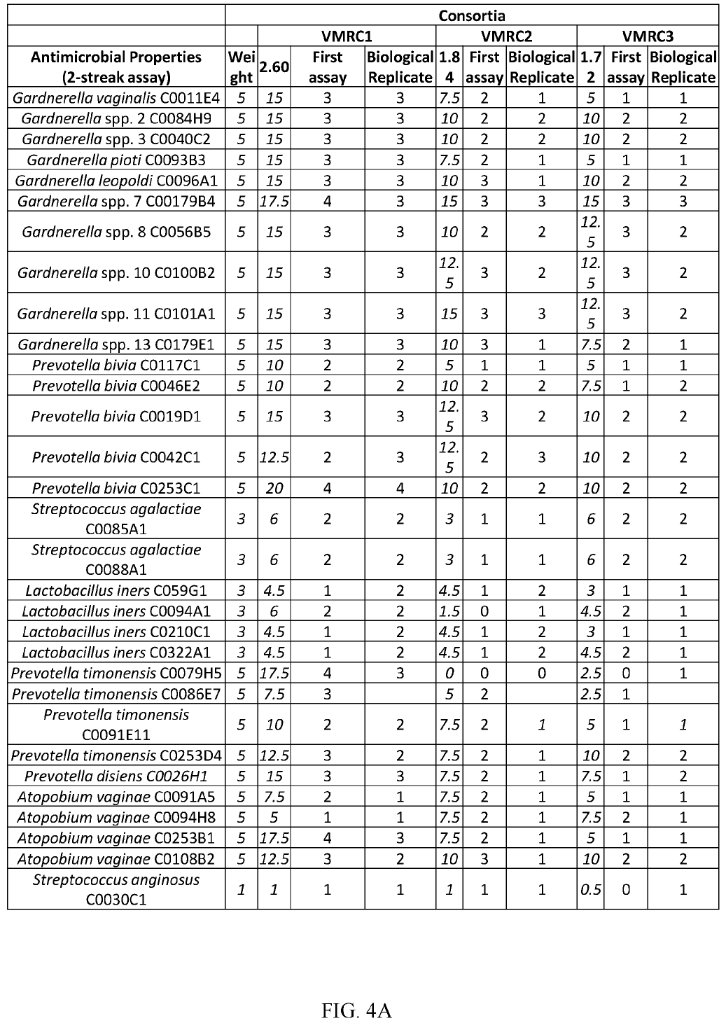

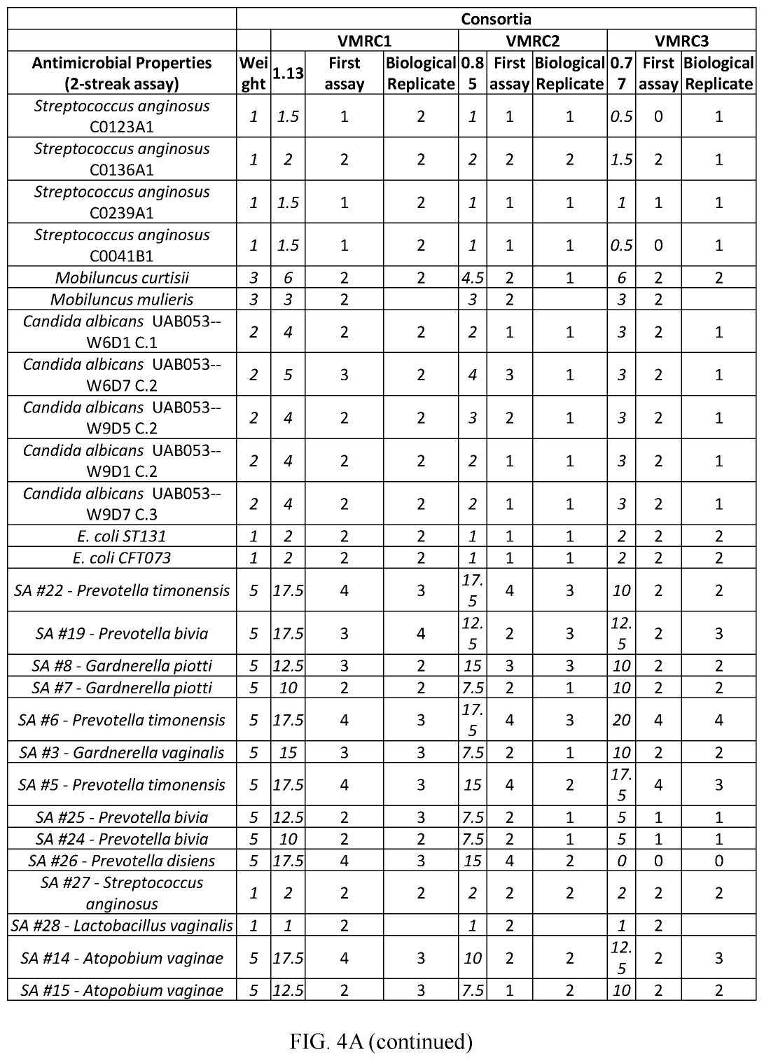

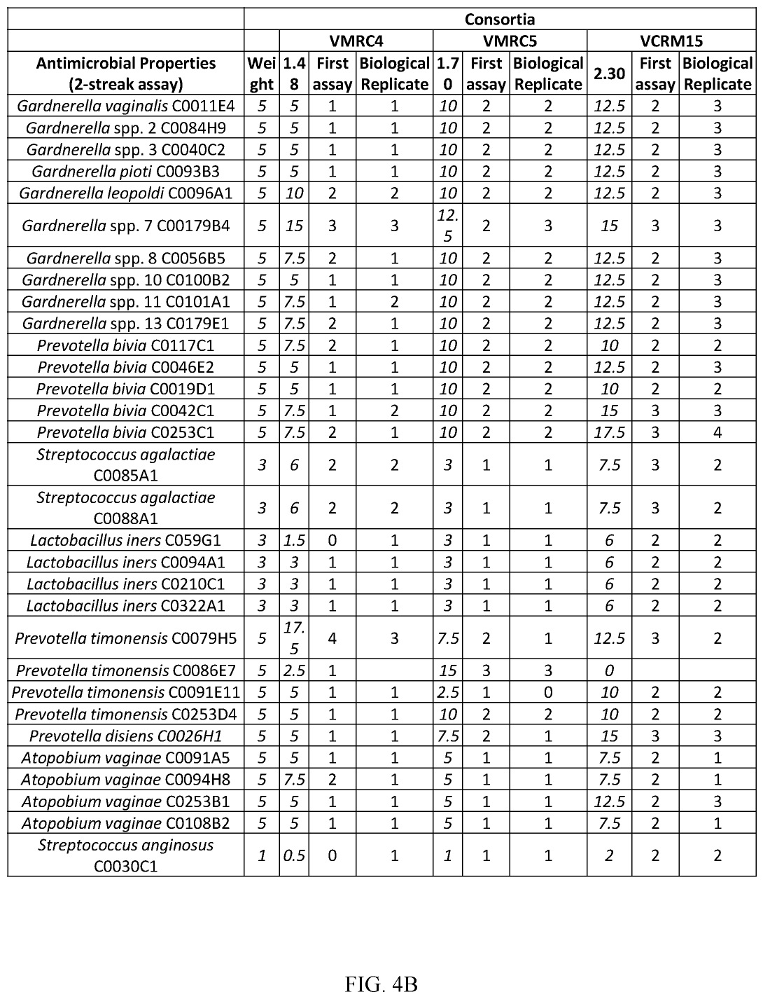

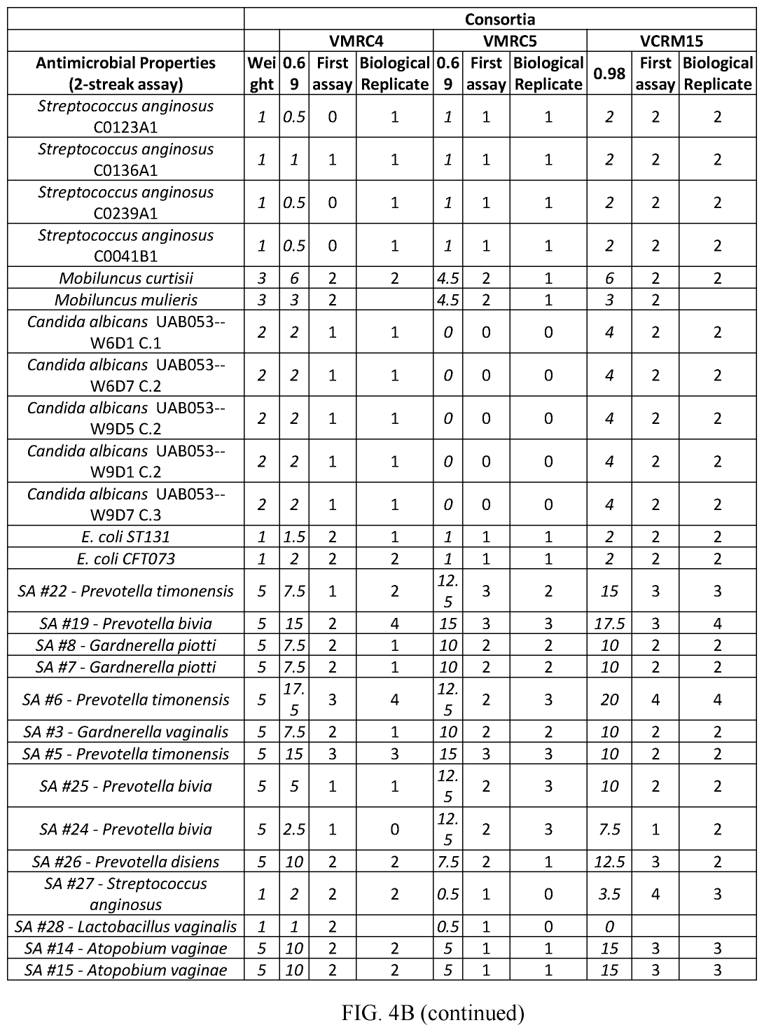

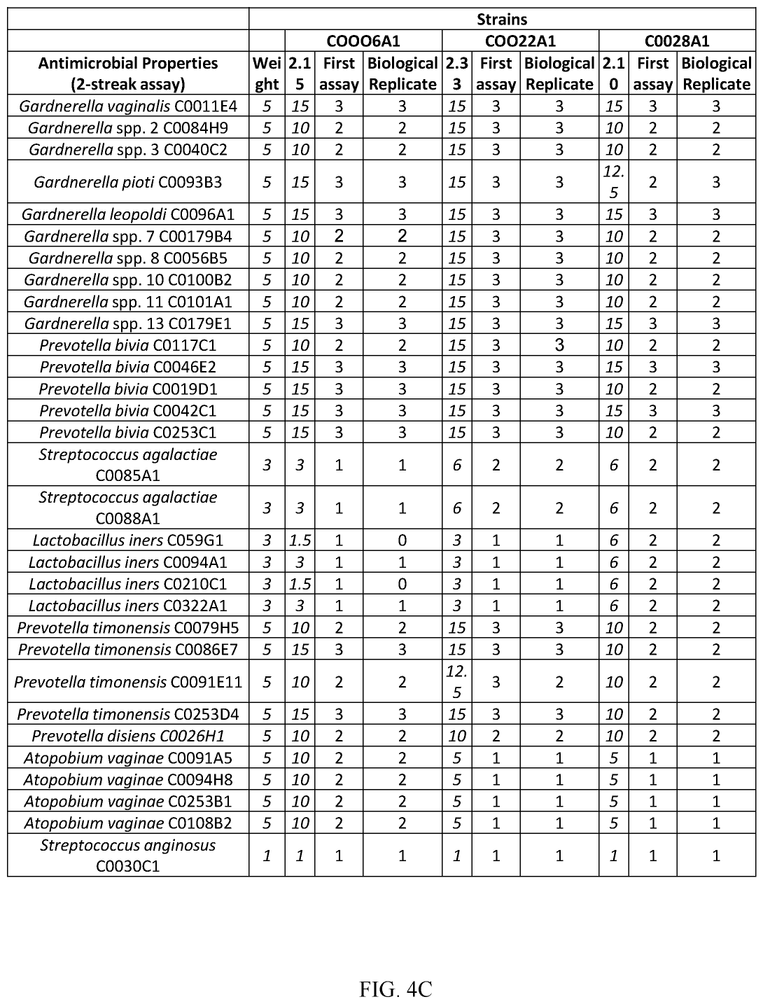

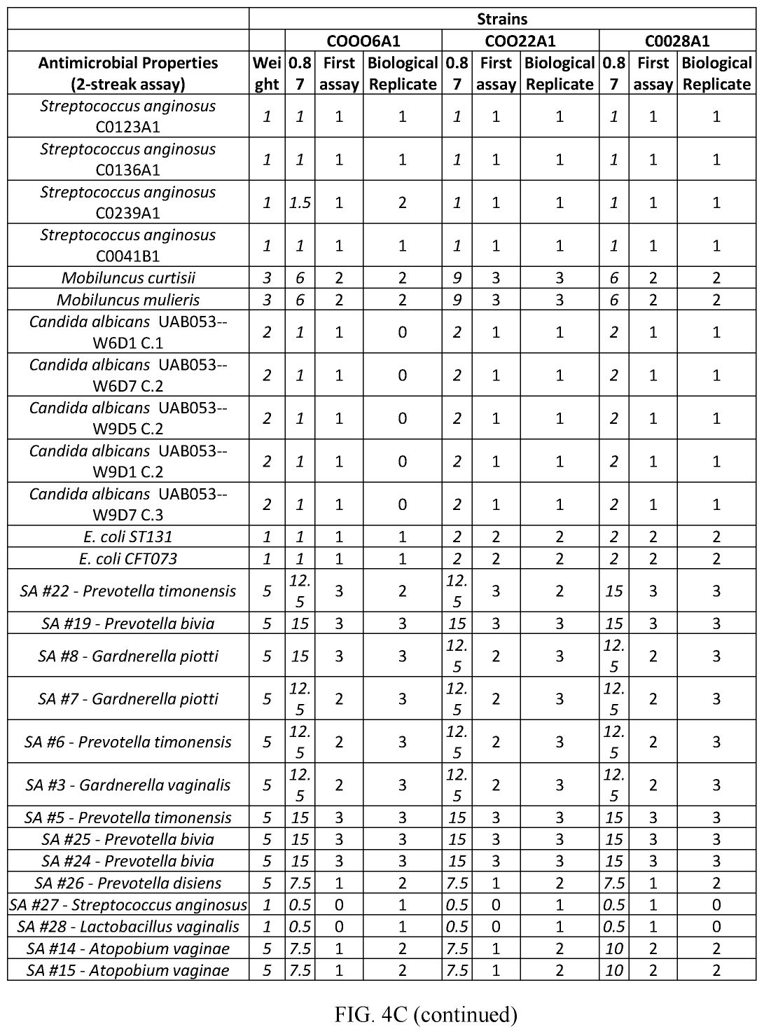

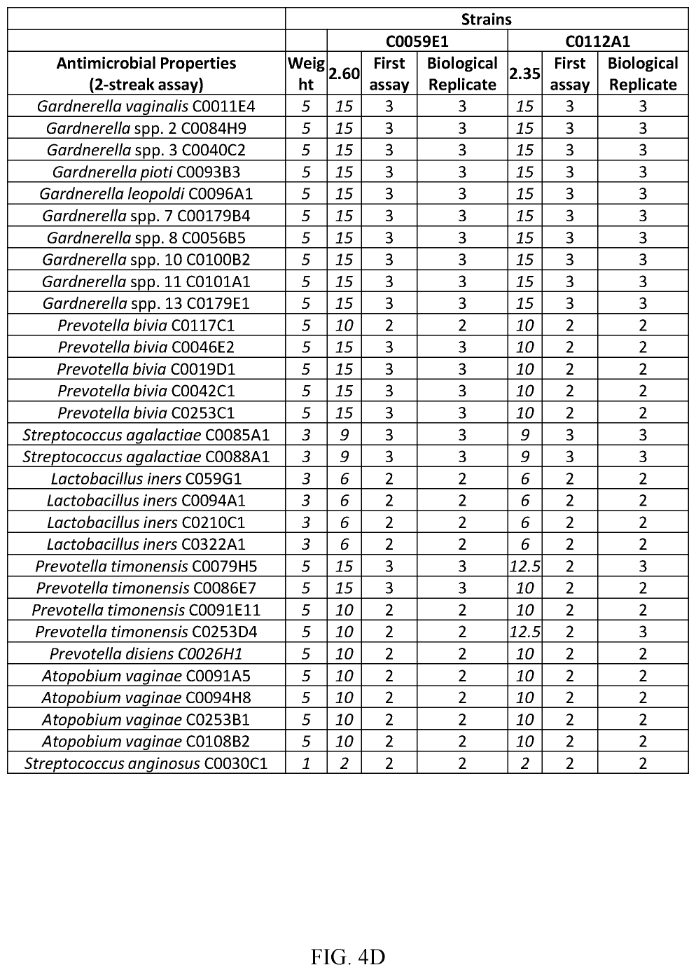

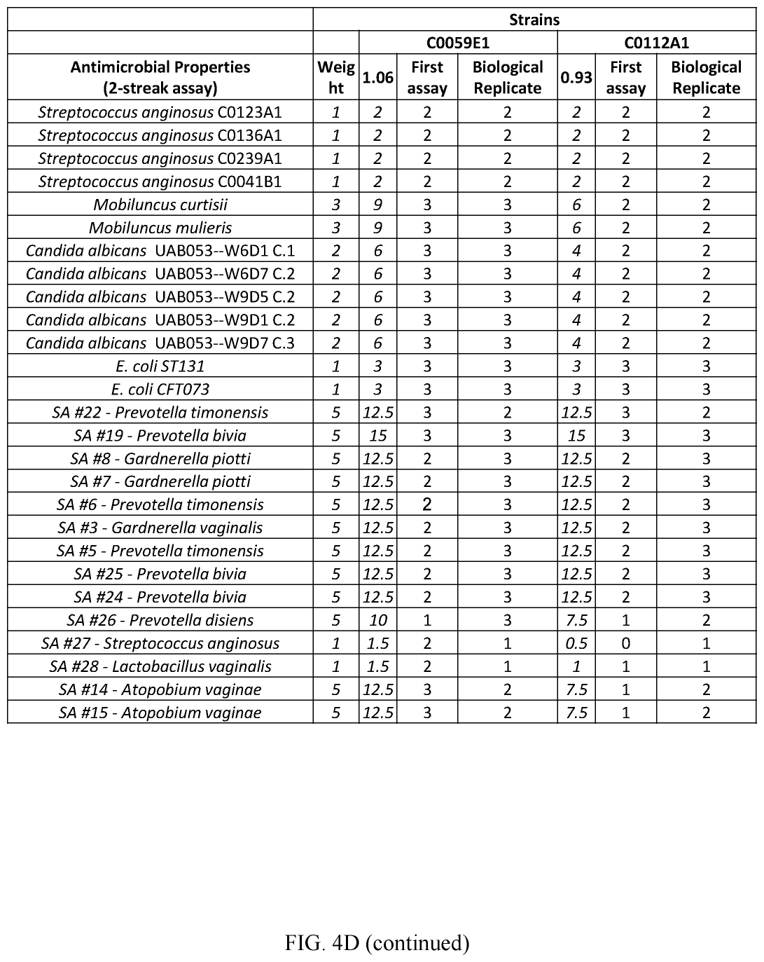

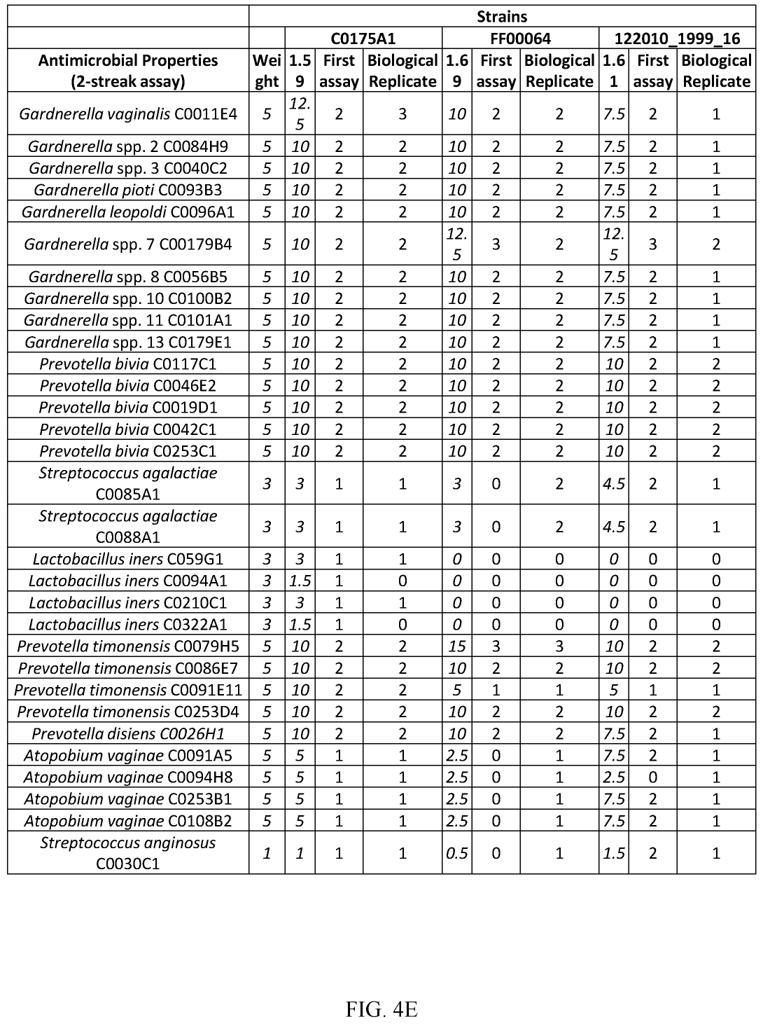

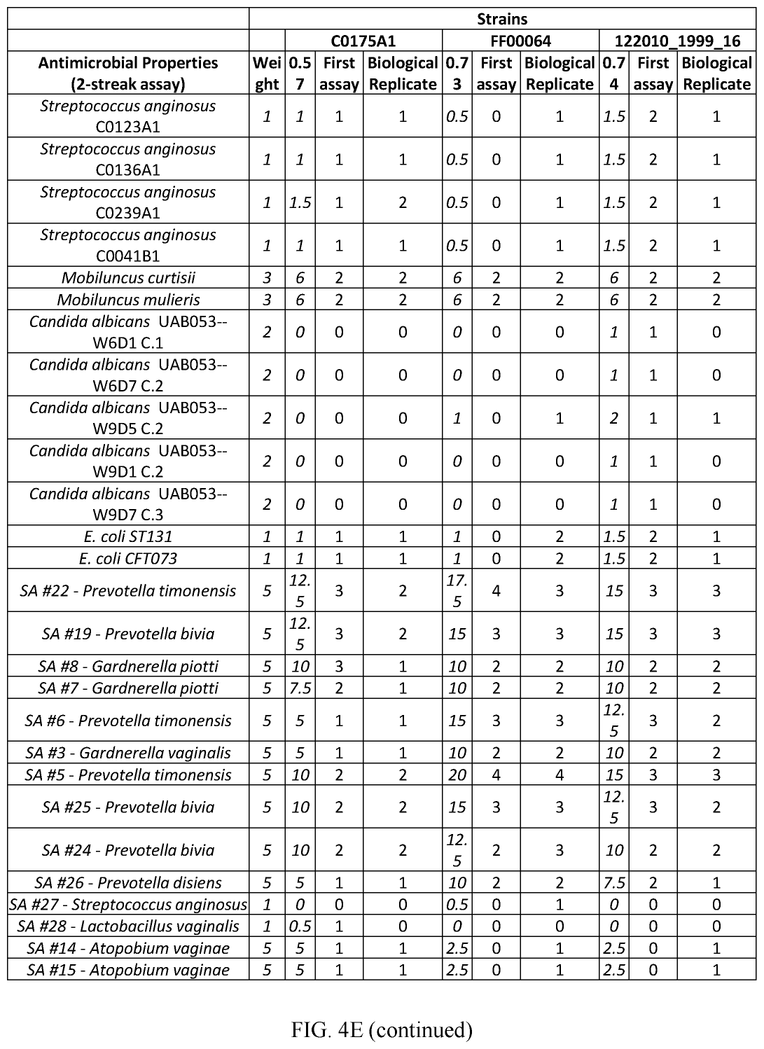

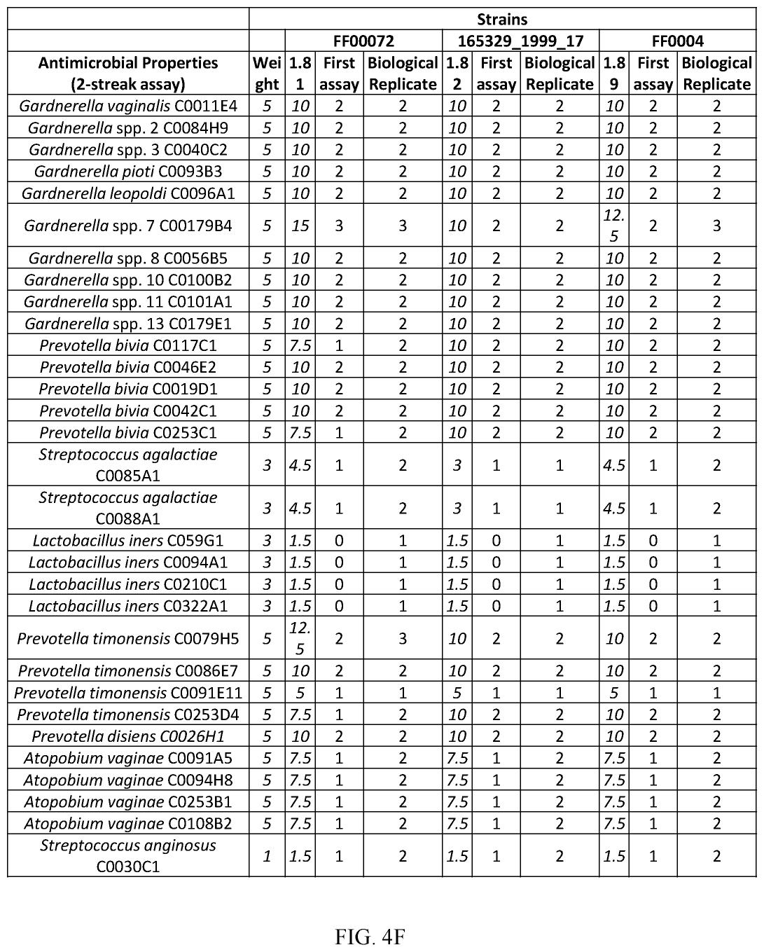

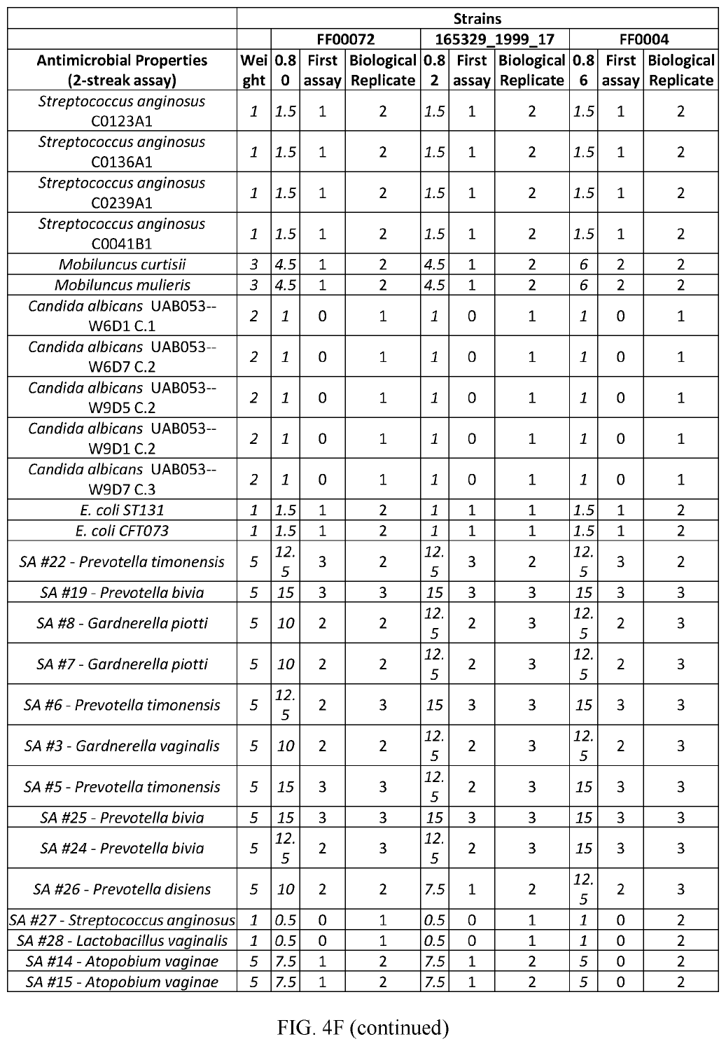

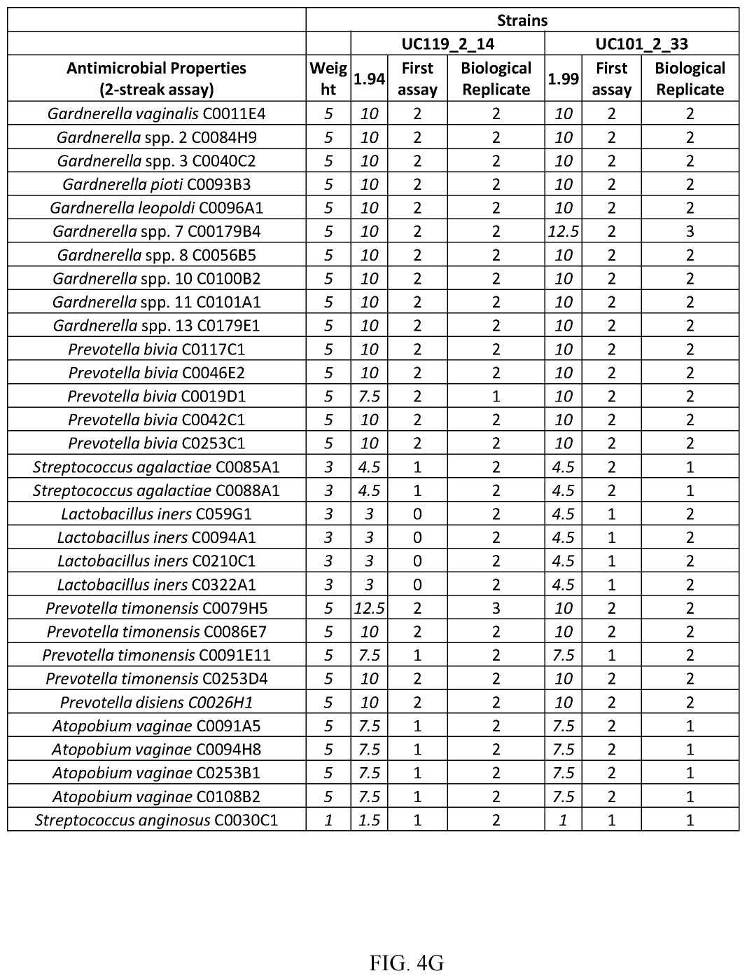

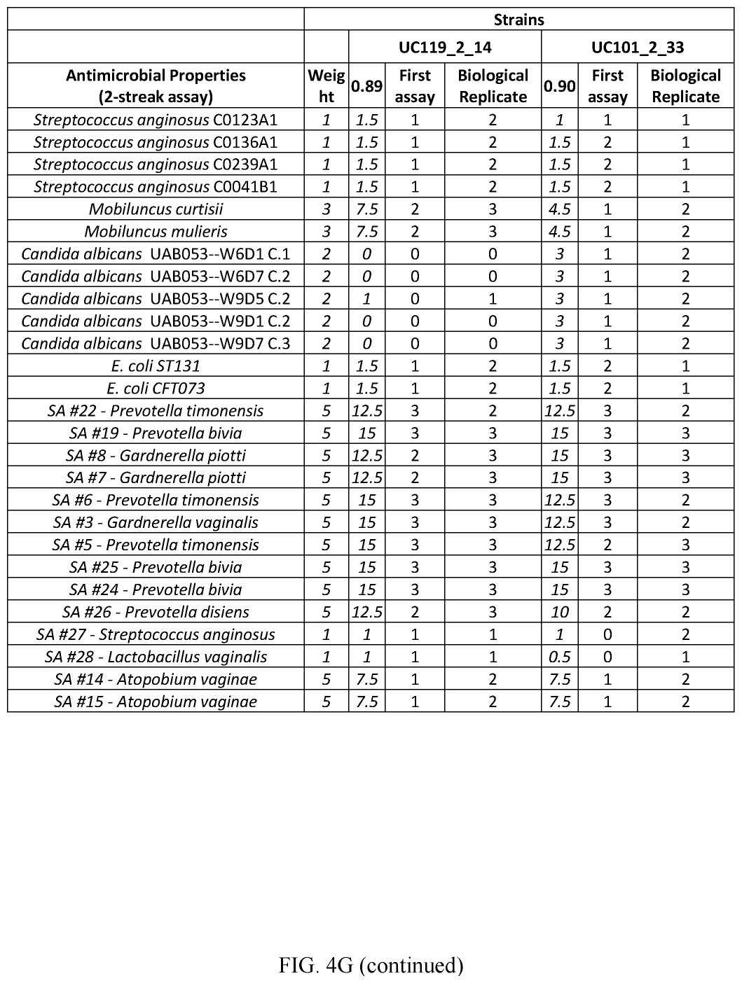

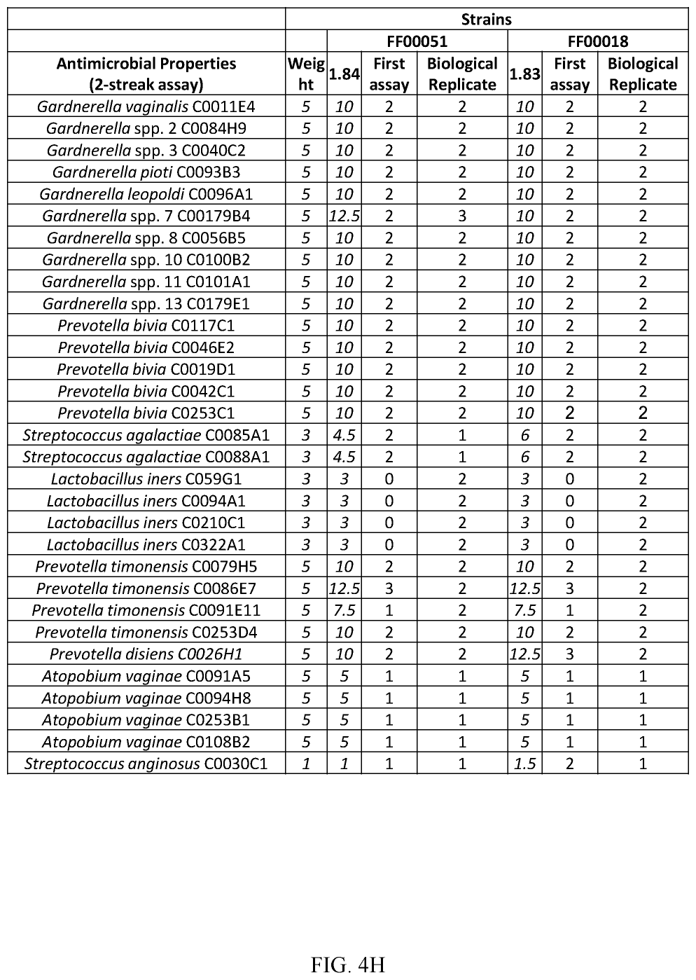

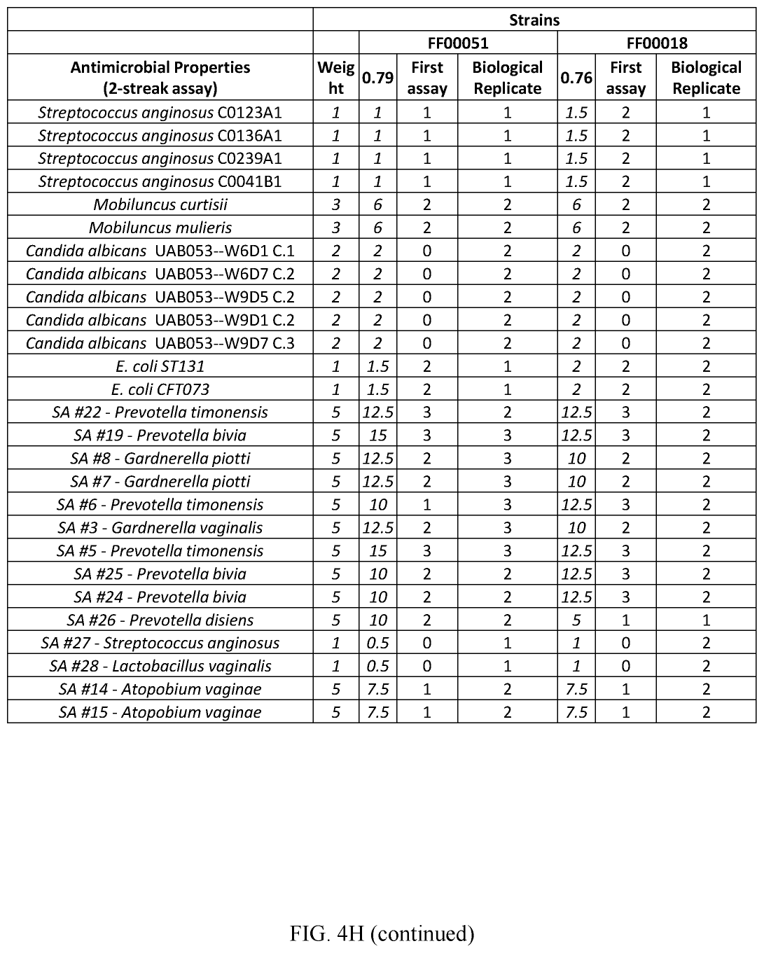

In some embodiments, the strains comprise or consist of a combination shown in FIG. 4A-4H.

Table 1 provides exemplary sequences that characterize these strains. In some embodiments, any of the strains can include additional plasmids. For example, strain 122010_1999_16 can include an additional plasmid (e.g., strain 122010_1999_16 plasmid of SEQ ID NO: 2). Exemplary strain genome sequences and plasmid sequences are listed in Table 1.

TABLE 1
Sequence Descriptions.

| SEQ | | |
|---|---|---|
| ID NO: | Description of Sequence | Strain |
| 1 | Strain 122010_1999_16 genome | Strain 122010_1999 |
| 2 | Strain 122010_1999_16 plasmid | Optional plasmid for Strain 122010_1999 |
| 3 | Strain C0006A1 genome | Strain C0006A1 |
| 4 | Strain C0006A1 plasmid | Optional plasmid for Strain C0006A1 |
| 5 | Strain C0175A1 genome | Strain C0175A1 |
| 6 | Strain C0175A1 plasmid | Optional plasmid for C0175A1 |
| 7 | Strain C0028A1 genome | Strain C0028A1 |
| 8 | Strain C0028A1 plasmid | Optional plasmid for Strain C0028A1 |
| 9 | Strain FF00018 genome | Strain FF00018 |
| 10 | Strain FF00018 plasmid 1 | Optional plasmid for Strain FF00018 |
| 11 | Strain FF00018 plasmid 2 | Optional plasmid for Strain FF00018 |
| 12 | Strain FF00072 genome | Strain FF00072 |
| 13 | Strain FF00072 plasmid | Optional plasmid for Strain FF00072 |
| 14 | Strain 185329_1999_17 genome | Strain 185329_1999_17 |
| 15 | Strain 185329_1999_17 plasmid | Optional plasmid for Strain 185329_1999_17 |
| 16 | Strain UC119_2_11 genome | Strain UC119_2_11 |
| 17 | Strain UC119_2_11 plasmid | Strain UC119_2_11 |
| 18 | Strain C0059E1 genome | Strain C0059E1 |
| 19 | Strain FF00004 genome | Strain FF00004 |
| 20 | Strain C0112A1 genome | Strain C0112A1 |
| 21 | Strain C0022A1 genome | Strain C0022A1 |
| 22 | Strain FF00064 genome | Strain FF00064 |
| 23 | Strain FF00051 genome | Strain FF00051 |
| 24 | Strain UC101_2_33 genome | Strain UC101_2_33 |

The following table contains additional information pertaining to strains described herein. In some embodiments, strains comprise sequences having 95% (e.g., 96%, 97%, 98%, 99%, 99.5%, 99.6%, 99.7%, 99.8%, 99.9%, 99.95%, or 99.99%) sequence identity to any of the sequences described in Table 1. In some embodiments, strains comprise sequences having 99.5% sequence identity to any of the sequences described in Table 1. In some embodiments, strains comprise sequences having 99.9% sequence identity to any of the sequences described in Table 1. In some embodiments, strains comprise any of the sequences described in Table 1.

The recitations “sequence identity,” “percent identity,” or for example, “comprising a sequence 95% identical to,” as used herein, refer to the extent that sequences are identical on an amino acid-by-amino acid basis, or a nucleotide-by-nucleotide basis, or over a window of comparison. Thus, a “percentage of sequence identity” can be calculated by comparing two optimally aligned sequences (e.g., nucleic acid) over the window of comparison, determining the number of positions at which the identical nucleic acid base (e.g., A, T, C, G, U) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

Calculations of sequence similarity or sequence identity between sequences (the terms are used interchangeably herein) can be performed as follows. To determine the percent identity of two amino acid sequences, or of two nucleic acid sequences, the sequences can be aligned for optimal comparison purposes (e.g., gaps can be introduced in one or both of a first and a second amino acid or nucleic acid sequence for optimal alignment and non-homologous sequences can be disregarded for comparison purposes. The comparison of sequences and determination of percent identity between two sequences can be accomplished using a mathematical algorithm. In some embodiments, the percent identity between two amino acid sequences is determined using the Needleman and Wunsch, (1970, J. Mol. Biol. 48:444-453) algorithm which has been incorporated into the GAP program in the GCG software package, using either a BLOSUM 62 matrix or a PAM250 matrix, and a gap weight of 16, 14, 12, 10, 8, 6, or 4 and a length weight of 1, 2, 3, 4, 5, or 6. 0), using a PAM120 weight residue table, a gap length penalty of 12 and a gap penalty of 4.

In some embodiments, the LBP composition comprises two or more (e.g., three, four, five, six, seven, eight, nine, ten, eleven, twelve, thirteen, fourteen, or fifteen) Lactobacillus crispatus strains selected from the group of 122010_1999_16, C0022A1, C0175A1, C0059E1, C0028A1, C0006A1, FF00018, UC101_2_33, UC119_2_11, 185329_1999_17, C0112A1, FF00004, FF00064, FF00051, and FF00072.

In some embodiments, the LBP composition comprises Lactobacillus crispatus strains C0022A1, C0175A1, C0059E1, UC101_2_33, FF00051, and FF00018.

In some embodiments, the LBP composition comprises Lactobacillus crispatus strains 122010_1999_16, C0022A1, C0175A1, C0059E1, C0028A1, C0006A1, FF00018, UC101_2_33, UC119_2_11, 185329_1999_17, C0112A1, FF00004, FF00064, FF00051, and FF00072.

In some embodiments, the LBP composition comprises Lactobacillus crispatus strains C0022A1, C0175A1, C0112A1, FF00051, FF00018, and UC101_2_33. In some embodiments, the LBP composition comprises Lactobacillus crispatus strains C0022A1, C0175A1, and UC101_2_33.

In some embodiments, the LBP composition comprises Lactobacillus crispatus strains C0175A1, C0112A1, FF00051, FF00018, and FF00064.

In some embodiments, the LBP composition comprises Lactobacillus crispatus strains C0006A1, C0112A1, C0175A1, FF00018, and FF00064.

In some embodiments, the vaginal live bio-therapeutic composition comprises two bacterial strains having at least about 95% sequence identity (e.g., at least about 96%, 97%, 98%, 99%, 99.5%, 99.9% or 100% sequence identity) to a Lactobacillus crispatus strain selected from FF00051, C0022A1, C0175A1, C0059E1, FF00018, UC101_2_33.

In some embodiments, the LBP composition comprises two or more (e.g., three, four, five, six, seven, eight, nine, ten, eleven, twelve, thirteen, fourteen, or fifteen) bacterial strains having at least about 95% sequence identity (e.g., at least about 96%, 97%, 98%, 99%, 99.5%, 99.9% or 100% sequence identity) to a Lactobacillus crispatus strain selected from the group of 122010_1999_16, C0022A1, C0175A1, C0059E1, C0028A1, C0006A1, FF00018, UC101_2_33, UC119_2_11, 185329_1999_17, C0112A1, FF00004, FF00064, FF00051, and FF00072.

In some embodiments, the only active agents in the compositions comprise or consist of Lactobacillus crispatus strains or isolates as described herein. Such compositions can be prepared in a manner well known in the pharmaceutical art and include at least one active agent, i.e., a strain as described herein.

In some embodiments, the L. crispatus strains are originally isolated from a vaginal sample (e.g., a tissue sample, a vaginal secretion sample, etc.) obtained from a female individual (or individual with a vagina) having a stable Lactobacillus-dominated vaginal microbiome. In some embodiments, the vaginal live bio-therapeutic composition further comprises at least one Lactobacillus crispatus strain originally isolated from a vaginal sample obtained from a female individual (or individual with a vagina) having a stable Lactobacillus-dominated vaginal microbiome. In some embodiments, the vaginal live bio-therapeutic composition further comprises at least one additional Lactobacillus crispatus strain originally isolated from a vaginal sample obtained from a female individual (or individual with a vagina) having a stable Lactobacillus-dominated vaginal microbiome.

LBPs as described herein can be combined with pharmaceutical excipients known in the art to enhance preservation and maintenance of the isolates prior to administration to produce pharmaceutical compositions or pharmaceutical formulations. Any of the pharmaceutical composition, pharmaceutical formulations, or unit doses described herein can be administered to an individual in need thereof.

In some embodiments, LBP compositions for vaginal administration can be formulated as suppositories (e.g., to be administered as suppositories). Exemplary suppositories can be prepared by mixing the particles with suitable non-irritating excipients or carriers such as cocoa butter, polyethylene glycol, or a suppository wax, which are solid at ambient temperature but liquid at body temperature and therefore melt in the vaginal cavity and release the LBPs.

In some embodiments, the LBPs can be formulated as a cream for topical administration. For example, LBPs can be formulated as a vulvo-vestibular cream. LBPs can be administered in a suspension in liquid form for use in a douche, in a capsule or vaginal tablet, or, for example, in dried form in a capsule or vaginal tablet. LBPs can be dried on film, for example preservation by vaporization (PBV), for vaginal administration in which the isolates will re-hydrate/reactivate in the vaginal environment. The means by which the composition comprising the LBPs described herein should be administered as is appropriate for the given composition.

In some embodiments the composition will be administered in a suspension in a vaginal capsule, or, for example, in dried form in a capsule. Methods for maintaining viability of L. crispatus isolates throughout the drying process are known to those of skill in the art. LBPs, including but not limited to dried preparations, can also be formulated in forms such that when administered, the isolates avoid killing and are only released to re-hydrate/reactivate in the relatively safer environment of the vagina. LBPs useful in the methods and compositions described herein can also be prepared and/or administered in admixture with one or more prebiotic compositions that promote the maintenance, establishment and/or growth of the probiotic.

Therapeutic compositions containing the LBPs for the treatment of vaginal dysbiosis can be conventionally administered in a unit dose. The term “unit dose” when used in reference to a therapeutic composition refers to physically discrete units suitable as unitary dosage for the individual, each unit containing a predetermined quantity of active material calculated to produce the desired therapeutic effect in association with the required physiologically acceptable diluent, i.e., carrier, or vehicle. Optimal dosing occurs over a duration of about two weeks or less.

LBP dosage forms, whether tablets, capsules, caplets, or particulates, may, if desired, be formulated so as to provide for controlled release of the therapeutic compositions, where controlled release may be sustained release, delayed release, or a combination thereof. Controlled release formulations are preferably sustained release, meaning gradual delivery of the therapeutic compositions over an extended time period. Generally, as will be appreciated by those of ordinary skill in the art, sustained release dosage forms are formulated by dispersing the live bacterial strains and other active agents/ingredients within a matrix of a gradually hydrolyzable material such as a hydrophilic polymer, or by coating a solid, drug-containing dosage form with such a material. Hydrophilic polymers useful for providing a sustained release coating or matrix include, by way of example: cellulosic polymers such as hydroxypropyl cellulose, hydroxyethyl cellulose, hydroxypropyl methyl cellulose, methyl cellulose, ethyl cellulose, cellulose acetate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate; and vinyl polymers and copolymers such as polyvinyl pyrrolidone, polyvinyl acetate, and ethylene-vinyl acetate copolymer.

In some embodiments, compositions as described herein can be conveniently provided as liquid preparations for vaginal administration, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Compositions described herein can be prepared as solutions. For example, solutions can be prepared by incorporating the LBPs utilized in the methods and compositions as described herein in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized.

The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, can be consulted to prepare suitable preparations, without undue experimentation.

In some embodiments, additional active agents are included in the compositions described and used herein. For example, a cysteine uptake inhibitor, an antibiotic treatment against BV, or a cysteine uptake inhibitor and an antibiotic treatment against BV. In some embodiments, a cysteine uptake inhibitor can prevent a bacterial cell (e.g., a cell of a bacterial species contributing to BV), from transporting cysteine into the cell from outside the cell. In some embodiments, a cysteine uptake inhibitor can selectively inhibit Lactobacillus iners growth in vitro or in vivo. Non-limiting cysteine uptake inhibitors can include S-methyl-L-cysteine (SMC) and seleno-DL-cysteine (SDLC). (See, for example, Bloom S M et al. Nat. Microbiol., (2022) March; 7 (3): 434-450, which is incorporated in its entirety.)

Various additives that enhance the stability and sterility of the compositions, including antimicrobial preservatives, antioxidants, chelating agents, and buffers, can be added. Prevention or reducing the risk of the action of microorganisms that contribute to vaginal dysbiosis or disorders associated with vaginal dysbiosis can be aided by various antibacterial and antifungal agents, for example, parabens, chlorobutanol, phenol, sorbic acid, and the like. In some embodiments, any of the additives that enhance the stability and sterility of the compositions do not inhibit the growth of L. crispatus, the survival of L. crispatus, or the growth of and the survival of L. crispatus.

The compositions can be isotonic, i.e., the compositions can have the same osmolarity and/or osmolality as blood and lacrimal fluid. The desired isotonicity of the compositions as described herein can be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Those skilled in the art will recognize that the components of the compositions should be selected to be chemically inert and will not affect the viability or efficacy of the isolates described in the present disclosure. This will present no problem to those skilled in chemical and pharmaceutical principles, or problems can be readily avoided by reference to standard texts or by simple experiments (not involving undue experimentation), from this disclosure and the documents cited herein.

The skilled artisan can readily determine the amount of isolates and optional additives, vehicles, and/or carrier in compositions and to be administered in methods as described herein. Of course, for any composition to be administered to a human, and for any particular method of administration, it is preferred to determine therefore: toxicity, the dosage of the composition(s), the concentration of components therein, and the timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

Optional additives include, but are not limited to, acids (e.g., lactic acid), carbon sources for L. crispatus (e.g., maltose and/or glycogen), L. crispatus cell wall materials or precursors (e.g., glutamate or glutamine), a buffer (e.g., magnesium citrate), mucoadhesives, antimicrobial peptides or bacteriophage that kill or impair the growth of other bacterial species that can limit L. crispatus growth, for example, Gardnerella vaginalis, Atopobium vaginae, Prevotella bivia, Megasphaera spp., and combinations thereof.

Maltose is intended to serve as a carbon and energy source for L. crispatus. Maltose is a common breakdown product of host-produced glycogen in the vaginal epithelium that L. crispatus is known to be capable of metabolizing. Maltose is also more accessible to L. crispatus than glycogen, which requires degradation prior to import into the cell. Maltose gel can promote L. crispatus maintenance in the vaginal of Rhesus macaques, where it otherwise does not easily colonize.

Glutamine is intended to serve as a precursor to glutamate, a building block for the L. crispatus cell wall. L. crispatus has a characteristically thick cell wall, which requires significant metabolic input to construct. Glutamine is used as an amine donor in peptidoglycan biosynthesis and in the amidation of aspartate residues in peptide cross-links. Glutamine is also not predicted to be transported by many of the other common non-Lactobacillus species including: Gardnerella spp., Atopobium spp., and Ca. lachnocurva vaginae. Metatranscriptomic data also indicate that unstable L. crispatus populations (those that are supplanted by other bacteria) exhibit higher expression of glutamine synthase, which converts glutamate back to glutamine. Supplementing glutamine can therefore help stabilize these populations.

Lactic acid, both D and L isoforms, are produced by L. crispatus as fermentation end products. By doing so, these bacteria engineer a low vaginal pH, thereby preventing, reducing, or limiting the growth of less desirable species. L. crispatus, and many other Lactobacillus species, are acid tolerant and capable of growth at lower pH. By supplementing with lactic acid, L. crispatus consortia will have a growth advantage by accessing nutrients with enzymes that are optimally set to operate at low pH over the other bacteria which are inhibited at low pH. Preclinical safety trials have been conducted for a vaginal ring device that released lactic acid.

Magnesium citrate is intended to serve as a pH buffer. As L. crispatus grows, it produces lactic acid, which lower vaginal pH. While low vaginal pH is thought be healthy, a vaginal pH that is too low may also prevent, reduce, or limit the growth of even the acid-tolerant L. crispatus. Citrate and citrate salts (e.g., magnesium citrate) can have an optimal buffering capacity between 3.0 and 6.2 and can help stabilize vaginal pH at about 4.2, thereby promoting L. crispatus growth while inhibiting growth of other anaerobic bacteria.

### Methods of Treatment

Methods described herein can include methods for the treatment or reducing risk of vaginal dysbiosis or having a vaginal dysbiosis disorder in an individual in need thereof using any of the compositions described herein.

As used in this context, to “treat” means to reduce the risk of developing a vaginal dysbiosis disorder, to reduce the frequency of a vaginal dysbiosis disorder, or to ameliorate at least one symptom of the disorder associated with vaginal dysbiosis disorders or a disorder associated with a vaginal dysbiosis disorder (e.g., bacterial vaginosis (BV), cervical dysplasia, preterm birth, acquiring sexually transmitted infections (e.g., HIV), post-operative gynecological infections, or unsuccessful in vitro fertilization and/or spontaneous conception) in an individual in need thereof. For example, a treatment can result in a reduction of the severity, the frequency, or the number of symptoms or a reduction of the risk of having symptoms associates with vaginal dysbiosis disorders (e.g., BV), or a disorder associated with a vaginal dysbiosis disorder (e.g., cervical dysplasia, preterm birth, acquiring sexually transmitted infections, post-operative gynecological infections, or unsuccessful in vitro fertilization and spontaneous conception) in an individual in need thereof.

In some embodiments, the individual is a female or has a vagina. In some embodiments, the individual is an otherwise healthy individual. In some embodiments, the individual is pregnant. In some embodiments, the individual is an otherwise healthy pregnant individual. In some embodiments, the individual is an individual having had 3 or fewer BV infections in the past 12 months. In some embodiments, the individual is an individual having had 3 or more BV infections in the past twelve months (or two in a six month period). In some embodiments, the individual is an individual with recurring BV. In some embodiments, the individual is an individual with frequent bv infections. Administration of a therapeutically effective amount of any of the compositions described herein for the treatment of any of the conditions described herein associated with vaginal dysbiosis can result in a reduction in the amount or severity of vaginal discharge with foul smelling odor, a reduction in the amount of grayish or white vaginal discharge, a reduction in a burning sensation that can occur while urinating, or a reduction in itchiness around the vaginal area, inter alia.

BV is a vaginal dysbiosis disorder characterized by, for example, a depletion of Lactobacilli in the vaginal bacterial population, an increased diversity of the vaginal bacterial population, and an elevated vaginal pH (i.e. above about 4.5 pH). BV can also refer to an individual with a depletion of L. crispatus and relatively high abundance of L. iners. BV can be identified using a variety of mechanisms. For example, BV can be identified using either the Nugent scoring system or the Amsel criteria. The Nugent scoring system uses vaginal smears that are plated on a microscopic slide in oil immersion, and a minimum of 10 high power fields are examined for three bacteria morphotypes: Lactobacillus, Gardnerella, and curved gram rods. Each of these three categories receives a score based on the number of bacteria counted. Subsequently, these three scores are added together for a total score ranging from 0-10. A score of 0-3 is negative for BV, a score of 4-6 is intermediate for BV, and a score of 7+ is positive for BV.

Alternatively, the Amsel criteria can be used. There are four parameters used to determine the presence or absence of BV. These are 1) thin, white, yellow, homogeneous discharge, 2) clue cells on wet mount microscopy, 3) a vaginal fluid pH greater than 4.5 when placing the discharge on litmus paper, and 4) a release fishy odor after adding 10% potassium hydroxide (KOH) solution to wet mount (also known as “whiff test”). A clue cell is a type of vaginal epithelial cell to which a cluster of bacteria is attached. At least two of the four symptoms are present to receive a positive BV test result. Preferably, at least three of the four symptoms of the Amsel criteria are present.

BV can also be identified or diagnoses using molecular techniques, and can be called a molecular diagnosis. A molecular diagnosis of BV identifies or diagnoses BV-like states using bacterial sequencing methods to identify vaginal dysbiosis.

BV can also be identified using various clinical diagnostic options such as an enzyme based tests (e.g., OSOM® BVBlue), DNA probe tests (e.g., BD Affirm™ VPIII), or multi-target PCR tests (e.g., BD Max™, GeneXpert®, or Aptima® BV assay).

In some embodiments, a method of treating bacterial vaginosis comprises or further comprises an alleviation of one or more symptoms of BV. In some embodiments, a method of treating BV comprises or further comprises an alleviation of one or more symptoms of BV. In some embodiments, the one or more symptoms include, but are not limited to, abnormal vaginal odor, abnormal vaginal discharge or a combination thereof. In some embodiments, alleviation refers to a lessening of the severity of one or more symptoms.

In some embodiments, the individual is a female (or has a vagina) presenting with an off-white (e.g., milky or gray), thin, homogeneous vaginal discharge, vaginal pH greater than or equal to 4.7, presence of clue cells of greater than or equal to 20% of the total epithelial cells on microscopic examination of a vaginal saline wet mount, a positive 10% KOH whiff test or a combination thereof. In some embodiments, the individual is a female (or has a vagina) presenting with an off-white (milky or gray), thin, homogeneous vaginal discharge, vaginal pH greater than or equal to 4.7, the presence of clue cells of greater than or equal to 20% of the total epithelial cells on microscopic examination of a vaginal saline wet mount, and a positive 10% KOH whiff test. In some embodiments, the individual is a female (or has a vagina) with confirmed BV. In some embodiments, BV is confirmed by the presence of four (4) Amsel criteria parameters selected from an off-white (milky or gray), thin, homogeneous vaginal discharge, vaginal pH greater than or equal to 4.7, the presence of clue cells of greater than or equal to 20% of the total epithelial cells on microscopic examination of a vaginal saline wet mount, a positive 10% KOH Whiff test and a gram stain slide Nugent score equal to, or higher than four (4) on bacterial analysis of vaginal samples. In some embodiments, the individual is a female (or has a vagina) with suspected BV. In some embodiments, suspected BV is indicated by the presence of four (4) Amsel criteria parameters selected from an off-white (milky or gray), thin, homogeneous vaginal discharge, vaginal pH greater than or equal to 4.7, the presence of clue cells of greater than or equal to 20%, of the total epithelial cells on microscopic examination of a vaginal saline wet mount, and a positive 10% KOH Whiff test. In some embodiments, the individual is a female (or has a vagina) presenting with an off-white (milky or gray), thin, homogeneous vaginal discharge, odor, or a combination thereof.

Some embodiments are directed to methods for preventing or reducing the risk recurrence of a vaginal dysbiosis disorder (e.g., BV), or a disorder associated with a vaginal dysbiosis disorder (e.g., cervical dysplasia, preterm birth, acquiring sexually transmitted infections, post-operative gynecological infections, or unsuccessful in vitro fertilization and spontaneous conception). Such methods of preventing or reducing the risk recurrence of a vaginal dysbiosis disorder a vaginal dysbiosis disorder (e.g., BV), or a disorder associated with a vaginal dysbiosis disorder (e.g., cervical dysplasia, preterm birth, acquiring sexually transmitted infections, post-operative gynecological infections, or unsuccessful in vitro fertilization and spontaneous conception) can comprise administering to an individual in need thereof any of the compositions described herein.

Some embodiments are directed to methods for restoring Lactobacillus dominated microbiota in an individual in need thereof. Such methods of restoring Lactobacillus dominated microbiota can comprise administering to an individual in need thereof any of the compositions described herein. The individual can have low Lactobacillus prevalence or no Lactobacillus in their vaginal microbiota. In some embodiments, a method for restoring Lactobacillus dominated microbiota in an individual in need thereof comprises administering a vaginal live bio-therapeutic composition to the individual. In some embodiments, a method for restoring Lactobacillus dominated microbiota in an individual in need thereof comprises administering a vaginal live bio-therapeutic composition to the individual wherein the composition comprises any of the compositions described herein. For example, a method for restoring Lactobacillus dominated microbiota in an individual in need thereof can comprise administering a vaginal live bio-therapeutic composition to the individual wherein the composition comprises two or more Lactobacillus crispatus strains selected from FF00051, C0022A1, C0175A1, C0059E1, FF00018, UC101_2_33, and combinations thereof, thereby restoring Lactobacillus dominated microbiota in the individual (see Table 1 for additional strain information).

In some embodiments, a method for restoring Lactobacillus dominated microbiota in an individual in need thereof comprises administering a vaginal live bio-therapeutic composition to the individual wherein the composition comprises any of the compositions described herein. For example, a method for restoring Lactobacillus dominated microbiota in an individual in need thereof comprises administering a vaginal live bio-therapeutic composition to the individual wherein the composition comprises two or more Lactobacillus crispatus strains selected from FF00051, C0022A1, C0175A1, C0059E1, FF00018, UC101_2_33, and combinations thereof, and optionally one or more additional Lactobacillus crispatus strains, thereby restoring Lactobacillus dominated microbiota in the individual. In some embodiments, a method for restoring Lactobacillus dominated microbiota in an individual in need thereof comprises administeringa vaginal live bio-therapeutic composition to the individual wherein the composition comprises two or more Lactobacillus crispatus strains selected from FF00051, C0022A1, C0175A1, C0059E1, FF00018, UC101_2_33, and combinations thereof, and at least one additional (e.g., one additional, two additional, three additional, four additional, five additional, six additional, seven additional, eight additional, nine additional, ten additional, eleven additional, twelve additional, thirteen additional, fourteen additional, or fifteen additional) Lactobacillus crispatus strain, thereby restoring Lactobacillus dominated microbiota in the individual. In some embodiments, a method for restoring Lactobacillus dominated microbiota in an individual in need thereof comprises administering a vaginal live bio-therapeutic composition to the individual wherein the composition comprises two or more Lactobacillus crispatus strains selected from FF00051, C0022A1, C0175A1, C0059E1, FF00018, UC101_2_33, and combinations thereof, and at least one an additional Lactobacillus crispatus strain selected from C0028A1, C0006A1, C0112A1, FF00004, UC119_2_11, FF00064, 122010_1999_16, 185329_1999_17, and FF00072, and combinations thereof, thereby restoring Lactobacillus dominated microbiota in the individual.

The present invention is additionally described by way of the following illustrative, non-limiting Examples that provide a better understanding of the present invention and of its many advantages.

### EXAMPLES

The following Examples illustrate some embodiments and aspects of the inventions described herein. It will be apparent to those skilled in the relevant art that various modifications, additions, substitutions, and the like can be performed without altering the spirit or scope of the inventions, and such modifications and variations are encompassed within the scope of the inventions as defined in the claims which follow. The following Examples do not in any way limit the inventions.

The Materials and Methods used in the following Examples are described in detail herein below.

### Example 1. Microbiome-Based Informed Method to Formulate Live Biotherapeutic Products

In a first phase, a collection of L. crispatus isolates were cultivated from vaginal swabs collected from South African and US women with stable L. crispatus-dominated vaginal microbiota. The genomes of these L. crispatus isolates were sequenced and the genes encoded in each of the genomes were combined into a custom database. In parallel, the metagenomes of several samples (3 to 5) from US and South African women were sequenced. These metagenomic sequence reads were then mapped to the VIRGO, a comprehensive catalogue of genes from the vaginal microbiome³⁶ affording the identification of all the genes and their taxonomic origin (the bacteria they belong to) in these metagenomes. For each metagenomic dataset, all genes assigned to L. crispatus were databased. As previously reported³⁶ the number of genes belonging to L. crispatus in a metagenome is higher than that encoded on one genome of L. crispatus, indicating that more than one strain of L. crispatus co-exist in a vaginal microbiome dominated by L. crispatus.

A total of 73 and 121 L. crispatus isolates were obtained from 6 US and 9 South African women with stable L. crispatus-dominated vaginal microbiome. In addition, a total of 44 and 23 metagenomes were obtained from 10 each US and South African women with stable L. crispatus-dominated vaginal microbiome. After dereplication of the L. crispatus genomes, a total of 15 isolate genomes remained to be included in the analysis.

The aim was to assemble a consortia of L. crispatus isolates which combined genes represent a maximum number of L. crispatus genes from each metagenome. To determine the optimal consortia of L. crispatus strains, a heuristic algorithm was utilized that matches genes encoded on isolate genomes to metagenomic data and determine the minimum number of L. crispatus strains while maximizing the number of L. crispatus genes encoded in the metagenomes from a maximum number of women. Gene accumulation curves were built for all combinations possible, that is, each time an additional strain was added to a consortium, the number of new genes matching the metagenome data were added to the total, and percentage of gene represented was plotted. The analysis showed that the number of genes added plateaued and was minimal after 6 strains and an average 75% of L. crispatus metagenome-encoded genes represented by the consortia. Essentially, the six selected strains represented the point at which the number of genes added by a 7th isolate was very low. The algorithm generated 335 combinations of 5 or 6 strains, representing 93 unique consortia of 5 or 6 strains. The consortia were then ranked based on the number of women they represented. Total of 5 consortia represented metagenomes from at least 4 women:
1. LC-106 (VMRC-1): A six-strain consortium comprising of L. crispatus strains C0022A1, C0175A1, C0059E1, FF00018, FF00051 and UC101_2_33 matched metagenomes from 7 women,
2. VMRC-2: A six-strain consortium comprising of L. crispatus strains C0022A1, C0175A1, C0112A1, FF00051, FF00018 and UC101_2_33 matched metagenomes from 4 women,
3. VMRC-3: A five-strain consortium comprising of L. crispatus strains C0022A1, C0175A1, C0059A1, UC119_2_14 and UC101_2_33 matched metagenomes from 6 women,
4. VMRC-4: A five-strain consortia comprising of L. crispatus strains C0175A1, C0112A1, FF00051, FF00018 and FF00064 matched metagenomes from 6 women,
5. VMRC-5: A five-strain consortia comprising of L. crispatus strains C0006A1, C0112A1, C0175A1, FF00018, and FF00064, matched metagenomes from 6 women.

Each of the consortia was further evaluated for 1) the potential inhibition of any of the strains comprised in these consortia by another one and; 2) for each consortia antimicrobial properties against a set of 12 non-desirable microbial species often found in the vagina of women with conditions such as BV. These included of total of 58 isolates from multiple strains (in parenthesis) of Gardnerella spp. (16), Prevotella bivia (8), Prevotella disiens (2), Prevotella timonensis (7), Atopobium vaginae (6), Streptococcus angiosus (6), Streptococcus agalactiae (2), Lactobacillus iners (4), Escherichia coli (2), Candida albicans (5), Mobiluncus mulieris (1) and Mobiluncus curtisii (1). Fourteen of these isolates were cultured from South African women.

None of the L. crispatus strains were shown to inhibit the growth of the other in cross-streaking assays on MRS agar plates. The antimicrobial assays were performed using a 2-streak assay, where the L. crispatus strains were streaked twice (¼ inch apart) across a 15×15 cm agar plates and incubated for 8 hours. Following incubation, a 20 μl suspension of the target strain was spotted in between the two L. crispatus streaks. A control 20 μl suspension of the same target strain was spotted on a separate plate and at the bottom of the 15×15 cm agar plate. The growth of the target strain was evaluated semi-quantitively and recorded using a 1-4 score system (1, indicating growth was similar to controls, i.e., no antimicrobial activity; and 4 indicating no growth and growth of the controls). The scored were tallied and a 1 to 5 weighing factor was applied to each target strains according to their importance to vaginal diseases. A score for the consortia was then calculated according to the following formula: Σ score*weight/Σ weights. Using this ranking system, LC-106 had the strongest antimicrobial properties.

The assay was performed for all 15 L. crispatus strains individually, and for each of the 5 consortia, and a consortium comprising all 15 strains. Consortia had higher antimicrobial properties than any individual strain (See FIGS. 4A-4H). Lastly, the genomes of all 15 strains were examined using the Comprehensive Antibiotic Resistance Database (CARD). 37 No antibiotic resistance genes were found on the genomes of these 15 L. crispatus strains. This was further confirmed by disc-assay (Etest) on MRS agar for azithromycin, doxycycline, clindamycin and amoxicillin, which showed no resistance. Because the genetic determinants for metronidazole resistance are unknown, metronidazole is a recommended treatment for several vaginal infection, and L. crispatus was expected to not be affected by metronidazole, we used a disc-assay to test for metronidazole and hydroxy-metronidazole resistance. The assay showed that the growth of all L. crispatus strains was unaffected by either metronidazole or hydroxy-metronidazole, while that of a metronidazole-sensitive G. vaginalis isolate was inhibited, and that of two metronidazole-resistant G. vaginalis isolates was not.

### Example 2. Lactobacillus crispatus Strains are not Inflammatory in Endocervical Cell Lines

Two assays were done to assess whether Lactobacillus crispatus strains comprising LBPs as described herein are inflammatory in Endocervical tissue. Either conditioned media in which the Lactobacillus crispatus strains were grown and applied to an endocervical cell line (End1) or heat killed Lactobacillus crispatus strains were cultured with End1 cells and secretion of inflammatory marker IL-8 was used as a readout of induced inflammation by End1 cells.

For the conditioned media assay, strains were grown in MRS broth overnight, and then seeded in new broth and grown for 5 hours to reach the exponential phase prior to collecting media.

For the heat-killed assay, broth culture was heated to 80° C. for 15 minutes, pelleted, resuspend and co-cultured for 20 hours with End1 cells in a 96-well plate, anaerobically.

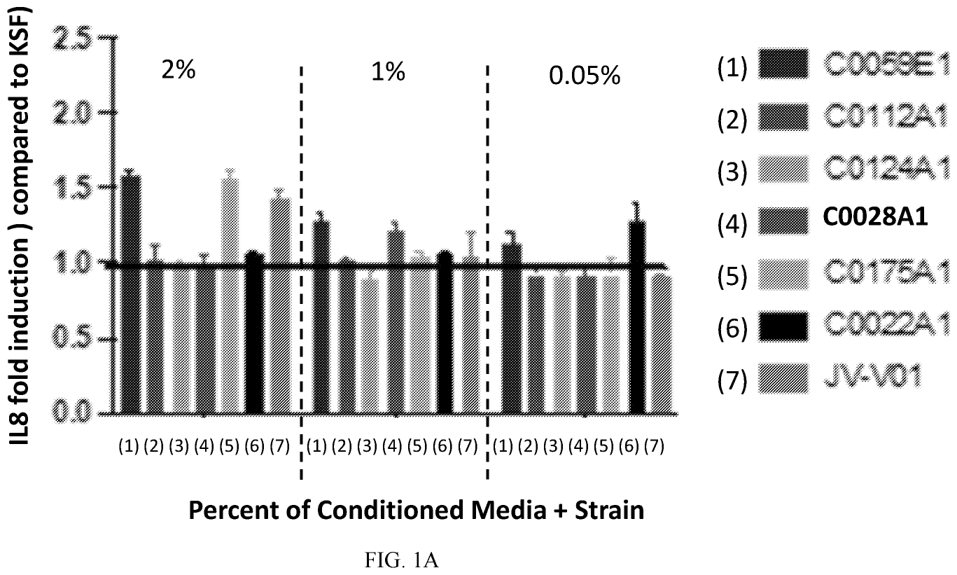

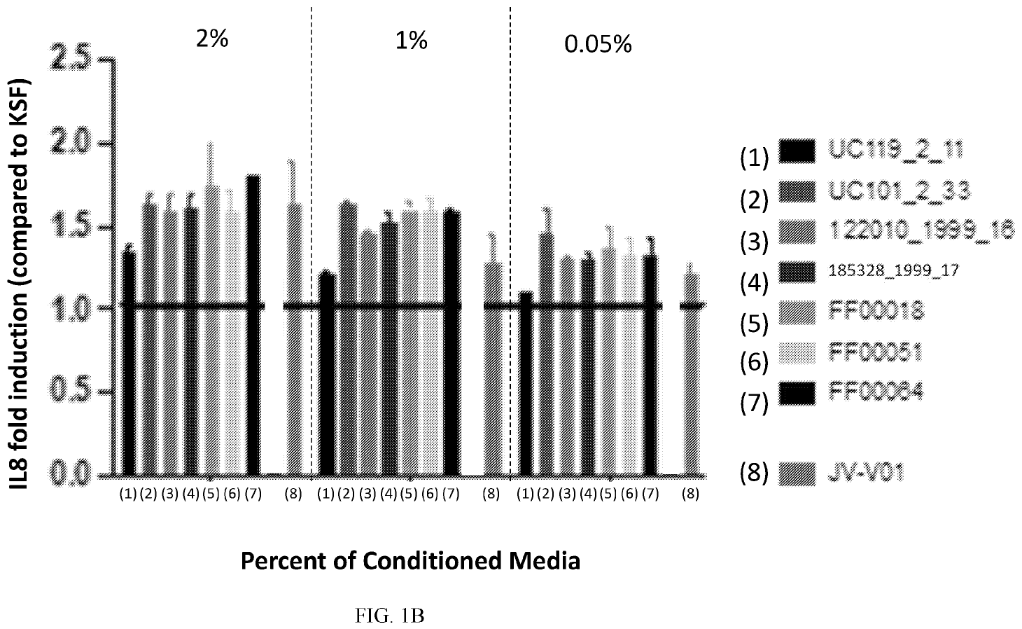

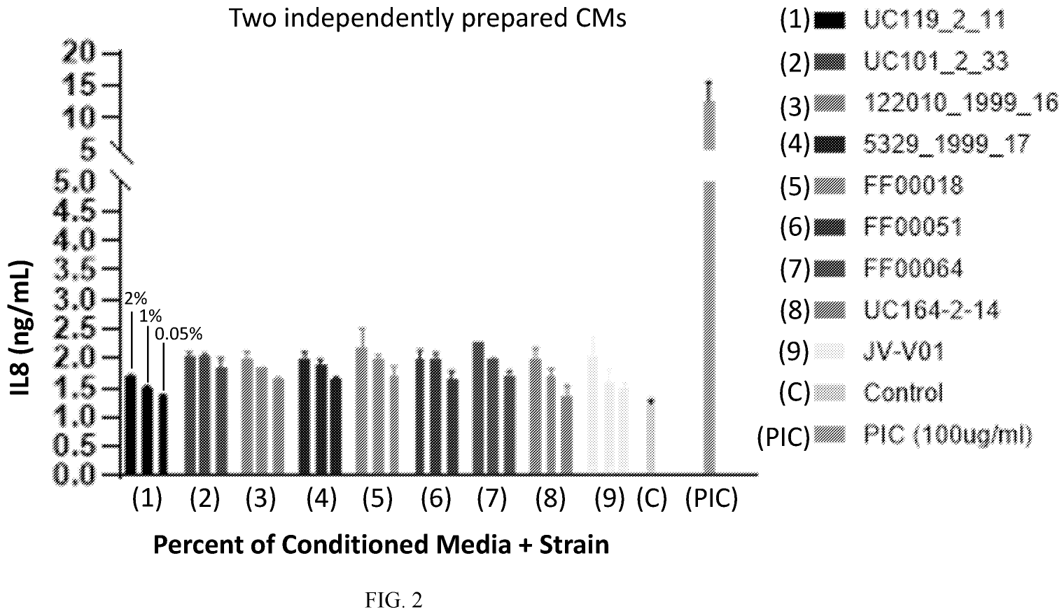

Using 2%, 1%, or 0.05% conditioned media, similar and low levels of IL-8 was observed between all strains (FIGS. 1A-1B, and FIG. 2).

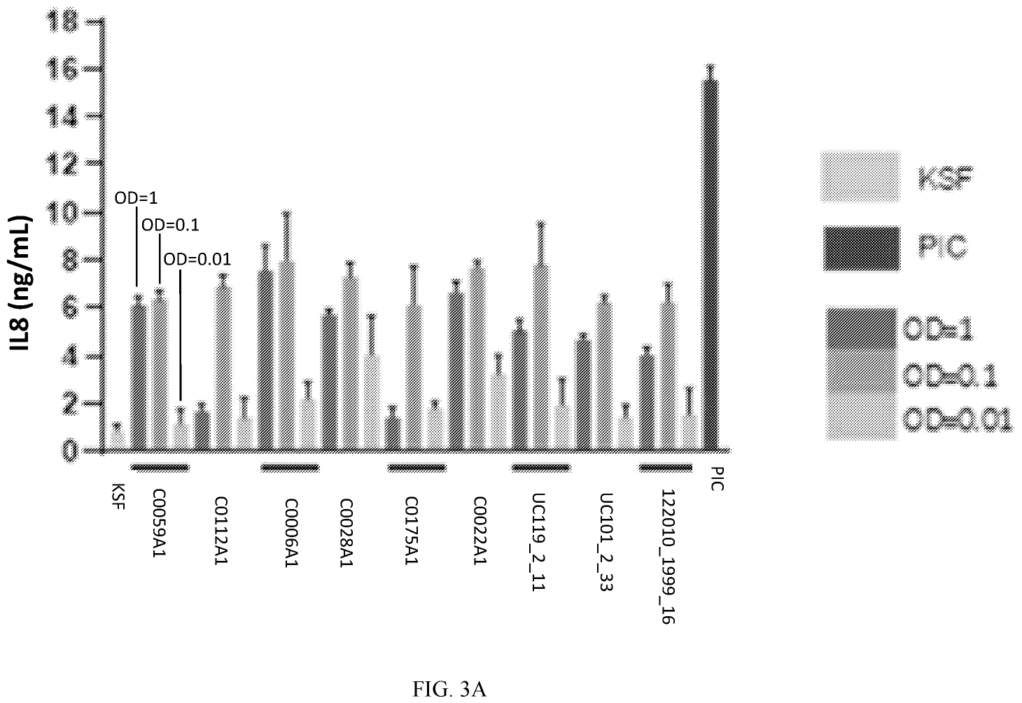

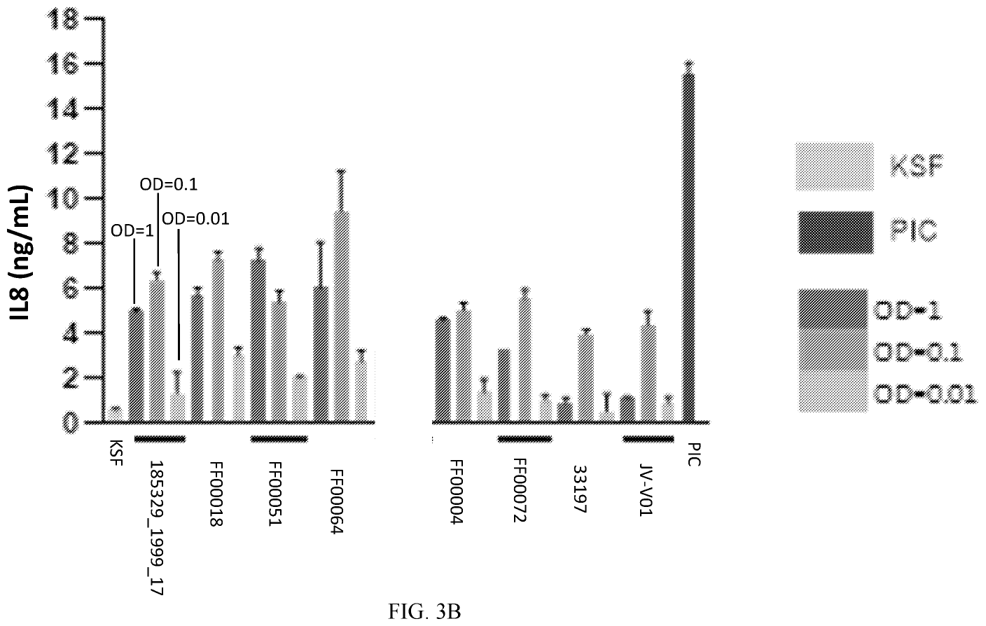

Culture of End1 cells with heat killed strains resulted in a dose response (i.e., OD1 shows more IL-8 than OD 0.1 and OD 0.01), with overall IL-8 levels remaining low (FIGS. 3A-3B). An OD of 10 corresponded to a MOI of 500-2000. An OD of 0.1 corresponded to a MOI of 50-200. An OD of 0.01 corresponded to a MOI of 5 to 20.

### Example 3. Phase 1 Trial of Multi-Strains L. crispatus Vaginal Live Biotherapeutic Products (LBP) is Proposed

A Phase I randomized trial of a novel live biotherapeutic intervention containing multiple strains of L. crispatus will compare safety and biologic effects of two different consortia of L. crispatus strains, and a variety of dosing strategies in women with BV who receive antibiotic treatment.

The primary objective of this study is 1) to determine colonization kinetics of a vaginally-delivered live biotherapeutic product, 2) to determine the capacity of the individual L. crispatus strains from the LBP to establish colonization in two geographically distinct populations and 3) inform the optimal dosing frequency and duration. As this intervention has not previously been tested in humans, and as there is no adequate animal model, a first human trial will be performed.

The study population comprises 50 pre-menopausal, non-pregnant individuals aged 18-40 years with BV diagnosed by Nugent score at two clinical sites: Boston, Massachusetts and KwaZulu Natal, South Africa. The treatment regimen will comprise the LC-106 LBP composition consisting of L. crispatus strains C0022A1, C0175A1, C0059E1, FF00051, FF00018 and UC101_2_33, the LC-115 LBP composition comprising or consisting of L. crispatus strains C0022A1, C0112A1, C0175A1, C0059E1, C0028A1, C0006A1, UC101_2_33, UC119_2_11, FF00004, FF00018, FF00064, FF00051, FF00072, 185329_1999_17 and 122010_1999_16, or any of the compositions described herein, concurrent with or following metronidazole treatment.
1. Placebo daily for 7d after metronidazole treatment
2. LC-106 daily for 7d after metronidazole treatment
3. LC-106 daily for 3d+4 days of placebo, starting after metronidazole treatment
4. LC-106 daily for 7d, starting on day 3 of metronidazole treatment
5. LC-115 daily for 7d after metronidazole treatment

Four study arms will have similar packaging, and participants and study staff will be blinded to their allocation: the 7-day placebo, and the 7 days of active LBP for LC-106 and LC-115 used after completion of antibiotics. In addition, for the individuals randomized to three days of active LBP, the LBP will be packaged with 4 days of placebo for a total of 7 days of study product. Those who are randomized to start LBP on day 3 of antibiotics will not be blinded.

Primary outcomes to be determined include safety, as measured by adverse event (AE) and serious adverse event (SA) reporting, and colonization with any L. crispatus strain contained in the live biotherapeutic product at 5 weeks after initiation of antibiotic therapy. Colonization will be defined as detection of any one of the L. crispatus strains contained in the LBP at >5% relative abundance on shotgun sequencing of the vaginal microbial community and a total of 10% of all L. crispatus strains.

Secondary outcomes to be determined include kinetics of colonization with any of the strains contained in the LBP throughout the study using strain-specific qPCR, BV by Nugent score (a gram stain scoring system for vaginal swabs to diagnose BV), non-iners Lactobacillus dominance of the vaginal microbiota, defined by >50% of sequences by 16S rRNA gene sequencing, relative abundance of non-iners Lactobacillus species and L. iners over time, change in diversity and composition of the vaginal microbial community over time, participant acceptability and tolerability of product and comparison of effects between US and South African sites.

Exploratory outcomes include, but are not limited to, change in vaginal fluid inflammatory markers, predictors of colonization, impact of baseline and post-antibiotic microbiota on response to intervention.

### REFERENCES

All patents, patent applications and publications mentioned in this specification are herein incorporated by reference to the same extent as if each independent patent and publication was specifically and individually indicated to be incorporated by reference.
1. Gosmann C, Anahtar M N, Handley S A, et al. Lactobacillus-Deficient Cervicovaginal Bacterial Communities Are Associated with Increased HIV Acquisition in Young South African Women. Immunity. Jan. 17, 2017; 46 (1): 29-37. doi: 10.1016/j.immuni.2016.12.013
2. Brotman R M, Shardell M D, Gajer P, et al. Interplay between the temporal dynamics of the vaginal microbiota and human papillomavirus detection. The Journal of Infectious Diseases. Dec. 1, 2014; 210 (11): 1723-33. doi: 10.1093/infdis/jiu330
3. Watts D H, Fazzari M, Minkoff H, et al. Effects of bacterial vaginosis and other genital infections on the natural history of human papillomavirus infection in HIV-1-infected and high-risk HIV-1-uninfected women. The Journal of Infectious Diseases. Apr. 1, 2005; 191 (7): 1129-39.
4. Hillier S L, Nugent R P, Eschenbach D A, et al. Association between bacterial vaginosis and preterm delivery of a low-birth-weight infant. The Vaginal Infections and Prematurity Study Group. The New England Journal of Medicine. Dec. 28, 1995; 333 (26): 1737-42.
5. Macones G A, Parry S, Elkousy M, Clothier B, Ural S H, Strauss J F, 3rd. A polymorphism in the promoter region of TNF and bacterial vaginosis: preliminary evidence of gene-environment interaction in the etiology of spontaneous preterm birth. American Journal of Obstetrics and Gynecology. June 2004; 190 (6): 1504-8; discussion 3A.
6. Gajer P, Brotman R M, Bai G, et al. Temporal dynamics of the human vaginal microbiota. Sci Transl Med. May 2, 2012; 4 (132): 132ra52. 7. Srinivasan S, Liu C, Mitchell C M, et al. Temporal variability of human vaginal bacteria and relationship with bacterial vaginosis. PLOS One. 2010; 5 (4): e10197. doi: 10.1371/journal.pone.0010197
8. Herbst-Kralovetz M M, Pyles R B, Ratner A J, Sycuro L K, Mitchell C. New Systems for Studying Intercellular Interactions in Bacterial Vaginosis. The Journal of Infectious Diseases. Aug. 15, 2016; 214 Suppl 1: S6-S13. doi: 10.1093/infdis/jiw130
9. Peebles K, Velloza J, Balkus J E, McClelland R S, Barnabas R V. High Global Burden and Costs of Bacterial Vaginosis: A Systematic Review and Meta-Analysis. Sexually Transmitted Diseases. May 2019; 46 (5): 304-311. doi: 10.1097/OLQ.0000000000000972
10. Fredricks D N, Fiedler T L, Thomas K K, Mitchell C M, Marrazzo J M. Changes in Vaginal Bacterial Concentrations with Intravaginal Metronidazole Therapy for Bacterial Vaginosis as Assessed by Quantitative PCR. Journal of Clinical Microbiology. 47 (3), 721-726.
11. Mitchell C, Balkus J, Agnew K, Lawler R, Hitti J. Changes in the vaginal microenvironment with metronidazole treatment for bacterial vaginosis in early pregnancy. Journal of Women's Health. November 2009; 18 (11): 1817-24. doi: 10.1089/jwh.2009.1378
12. Muzny C A, Kardas P. A Narrative Review of Current Challenges in the Diagnosis and Management of Bacterial Vaginosis. Sexually Transmitted Diseases. 2020 July: 47 (7): 441, doi: 10.1097/OLQ.0000000000001178
13. Petrova M I, Reid G, Vaneechoutte M, Lebeer S. Lactobacillus iners: Friend or Foe? Trends Microbiol. March 2017; 25 (3): 182-191. doi: 10.1016/j.tim.2016.11.007
14. Ravel J, Gajer P, Abdo Z, et al. Vaginal microbiome of reproductive-age women. Proc Natl Acad Sci USA. Mar. 15, 2010; 108 Supplment 1:4680-7. doi: 1002611107
15. Lambert J A, John S, Sobel J D, Akins R A. Longitudinal analysis of vaginal microbiome dynamics in women with recurrent bacterial vaginosis: recognition of the conversion process. PLOS One. 2013; 8 (12): e82599. doi: 10.1371/journal.pone.0082599
16. Mayer B T, Srinivasan S, Fiedler T L, Marrazzo J M, Fredricks D N, Schiffer J T. Rapid and Profound Shifts in the Vaginal Microbiota Following Antibiotic Treatment for Bacterial Vaginosis. The Journal of Infectious Diseases. Sep. 1, 2015; 212 (5): 793-802. doi: 10.1093/infdis/jiv079
17. Ravel J, Brotman R M, Gajer P, et al. Daily temporal dynamics of vaginal microbiota before, during and after episodes of bacterial vaginosis. Microbiome. 2013; 1 (1): 29. doi: 10.1186/2049-2618 Jan. 29
18. van de Wijgert J, Verwijs M C. Lactobacilli-containing vaginal probiotics to cure or prevent bacterial or fungal vaginal dysbiosis: a systematic review and recommendations for future trial designs. BJOG. January 2020; 127 (2): 287-299. doi: 10.1111/1471-0528.15870
19. Verwijs M C, Agaba S K, Darby A C, van de Wijgert J. Impact of oral metronidazole treatment on the vaginal microbiota and correlates of treatment failure. American Journal of Obstetrics and Gynecology. Aug. 9, 2019; doi: 10.1016/j.ajog.2019.08.008
20. Hemmerling A, Harrison W, Schroeder A, et al. Phase 2a study assessing colonization efficiency, safety, and acceptability of Lactobacillus crispatus CTV-05 in women with bacterial vaginosis. Sexually Transmitted Diseases. December 2010; 37 (12): 745-50. doi: 10.1097/OLQ.0b013e3181e50026
21. Ngugi B M, Hemmerling A, Bukusi E A, et al. Effects of bacterial vaginosis-associated bacteria and sexual intercourse on vaginal colonization with the probiotic Lactobacillus crispatus CTV-05. Sexually Transmitted Diseases. November 2011; 38 (11): 1020-7. doi: 10.1097/OLQ.0b013e3182267ac4
22. Cohen C R, Wierzbicki M R, French A L, et al. Randomized Trial of Lactin-V to Prevent Recurrence of Bacterial Vaginosis. The New England Journal of Medicine. May 14, 2020; 382 (20): 1906-1915. doi: 10.1056/NEJMoa1915254
23. Bilardi J E, Walker S, Temple-Smith M, et al. The burden of bacterial vaginosis: women's experience of the physical, emotional, sexual and social impact of living with recurrent bacterial vaginosis. PLOS One. 2013; 8 (9): e74378. doi: 10.1371/journal.pone.0074378
24. DiGiulio D B, Romero R, Amogan H P, et al. Microbial prevalence, diversity and abundance in amniotic fluid during preterm labor: a molecular and culture-based investigation. PLOS One. 2008; 3 (8): e3056. doi: 10.1371/journal.pone.0003056
25. Hay P E, Lamont R F, Taylor-Robinson D, Morgan D J, Ison C, Pearson J. Abnormal bacterial colonisation of the genital tract and subsequent preterm delivery and late miscarriage. BMJ (Clinical research ed). Jan. 29, 1994; 308 (6924): 295-8.
26. Watts D H, Krohn M A, Hillier S L, Eschenbach D A. Bacterial vaginosis as a risk factor for post-cesarean endometritis. Obstetrics and Gynecology. January 1990; 75 (1): 52-8.
27. Moore D E, Soules M R, Klein N A, Fujimoto V Y, Agnew K J, Eschenbach D A. Bacteria in the transfer catheter tip influence the live-birth rate after in vitro fertilization. Fertility and Sterility. December 2000; 74 (6): 1118-24.
28. Boeke A J, Dekker J H, van Eijk J T, Kostense P J, Bezemer P D. Effect of lactic acid suppositories compared with oral metronidazole and placebo in bacterial vaginosis: a randomised clinical trial. Genitourinary Medicine. October 1993; 69 (5): 388-92.
29. Bradshaw C S, Morton A N, Hocking J, et al. High recurrence rates of bacterial vaginosis over the course of 12 months after oral metronidazole therapy and factors associated with recurrence. The Journal of Infectious Diseases. Jun. 1, 2006; 193 (11): 1478-86.
30. Bradshaw C S, Pirotta M, De Guingand D, et al. Efficacy of oral metronidazole with vaginal clindamycin or vaginal probiotic for bacterial vaginosis: randomised placebo-controlled double-blind trial. PLOS One. 2012; 7 (4): e34540. doi: 10.1371/journal.pone.0034540
31. Bohbot J M, Vicaut E, Fagnen D, Brauman M. Treatment of bacterial vaginosis: a multicenter, double-blind, double-dummy, randomised phase III study comparing secnidazole and metronidazole. Infectious Diseases in Obstetrics and Gynecology. 2010 doi: 10.1155/2010/705692
32. Brandt M, Abels C, May T, Lohmann K, Schmidts-Winkler I, Hoyme U B. Intravaginally applied metronidazole is as effective as orally applied in the treatment of bacterial vaginosis, but exhibits significantly less side effects. European Journal of Obstetrics, Gynecology, and Reproductive Biology. December 2008; 141 (2): 158-62. doi: 10.1016/j.ejogrb.2008.07.022
33. Schwebke J R, Lensing S Y, Lee J, et al. Treatment of Male Sexual Partners of Women with Bacterial Vaginosis (BV); A Randomized, Double-Blind, Placebo-Controlled Trial. Clin Infect Dis. Dec. 31, 2020; doi: 10.1093/cid/ciaa1903
34. Turner A N, Carr Reese P, Fields K S, et al. A blinded, randomized controlled trial of high-dose vitamin D supplementation to reduce recurrence of bacterial vaginosis. American Journal of Obstetrics and Gynecology. November 2014; 211 (5): 479 e1-479 e13. doi: 10.1016/j.ajog.2014.06.023
35. Ratten L K, Plummer E L, Murray G L, et al. Sex is associated with the persistence of non-optimal vaginal microbiota following treatment for bacterial vaginosis: a prospective cohort study. BJOG. March 2021; 128 (4): 756-767. doi: 10.1111/1471-0528.16430
36. Ma, B. et al. A comprehensive non-redundant gene catalog reveals extensive within-community intraspecies diversity in the human vagina. Nature Communications 11, 940-13 (2020).
37. Alcock et al. 2020. CARD 2020: Antibiotic Resistome Surveillance with the Comprehensive Antibiotic Resistance Database. Nucleic Acids Research, 48, D517-D525.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating or reducing the risk of vaginal dysbiosis in an individual in need thereof, said method comprising administering a vaginal live bio-therapeutic composition to the individual, wherein the composition comprises two or more Lactobacillus crispatus strains selected from FF00051, C0022A1, C0175A1, C0059E1, FF00018, UC101_2_33 and combinations thereof, thereby treating vaginal dysbiosis in the individual.

2. The method of claim 1, wherein the vaginal live bio-therapeutic composition further comprises at least one additional Lactobacillus crispatus strain originally isolated from a vaginal tissue sample obtained from a female individual or individual with a vagina having a stable Lactobacillus-dominated vaginal microbiome.

3. The method of claim 2, wherein the at least one additional Lactobacillus crispatus strain is selected from C0028A1, C0006A1, C0112A1, FF00004, UC119_2_11, FF00064, 122010_1999_16, 185329_1999_17 and FF00072 and combinations thereof.

4. The method of claim 1, where in the vaginal dysbiosis is bacterial vaginosis.

5. A vaginal live bio-therapeutic composition comprising two or more Lactobacillus crispatus strains selected from FF00051, C0022A1, C0175A1, C0059E1, FF00018, UC101_2_33 and combinations thereof.

6. The vaginal live bio-therapeutic composition of claim 5, wherein the composition further comprises at least one additional Lactobacillus crispatus strain originally isolated from a vaginal tissue sample obtained from a female individual or individual with a vagina having a stable Lactobacillus-dominated vaginal microbiome.

7. The vaginal live bio-therapeutic composition of claim 6, wherein the at least one additional Lactobacillus crispatus strain is selected from C0028A1, C0006A1, C0112A1, FF00004, UC119_2_11, FF00064, 122010_1999_16, 185329_1999_17, and FF00072 and combinations thereof.

8. The vaginal live bio-therapeutic composition of claim 6, wherein the composition further comprises at least one of lactic acid, maltose, glycogen, glutamine and magnesium citrate or combinations thereof.

9. The vaginal live bio-therapeutic composition of claim 6, wherein the composition further comprises at least one mucoadhesive.

10. The vaginal live bio-therapeutic composition of claim 6, wherein the composition further comprises at least one antimicrobial peptide.

11. The vaginal live bio-therapeutic composition of claim 6, wherein the composition further comprises at least one cysteine uptake inhibitor.

12. A method for restoring Lactobacillus dominated microbiota in an individual in need thereof, said method comprising administering a vaginal live bio-therapeutic composition to the individual, wherein the composition comprises two or more Lactobacillus crispatus strains selected from FF00051, C0022A1, C0175A1, C0059E1, FF00018, UC101_2_33 and combinations thereof, thereby restoring Lactobacillus dominated microbiota in the individual.

13. The method of claim 12, wherein the vaginal live bio-therapeutic composition further comprises at least one additional Lactobacillus crispatus strain originally isolated from a vaginal tissue sample obtained from a female individual or individual with a vagina having a stable Lactobacillus-dominated vaginal microbiome.

14. The method of claim 13, wherein the at least one additional Lactobacillus crispatus strain is selected from C0028A1, C0006A1, C0112A1, FF00004, UC119_2_11, FF00064, 122010_1999_16, 185329_1999_17 and FF00072 and combinations thereof.
